SEQUENCE LISTING (8 sequences)

SEQ ID NO: 1 - protein, 12 aa, Artificial Sequence
  Artificial C-terminal amide peptide of Met-12
  MOD_RES (12)..(12): AMIDATION
HHIYLGAVNYIY

SEQ ID NO: 2 - protein, 12 aa, Artificial Sequence
  Artificial derivative of Met-12
  MOD_RES (1)..(6): Desamino modification
  MOD_RES (8)..(12): Desamino modification
YIYNVAGLYIHH

SEQ ID NO: 3 - protein, 12 aa, Artificial Sequence
  Artificial derivative of C-terminal amide peptide of Met-12
  MOD_RES (1)..(12): Desamino modification
  MOD_RES (12)..(12): AMIDATION
YIYNVAGLYIHH

SEQ ID NO: 4 - protein, 12 aa, Artificial Sequence
  Artificial derivative of C-terminal amide peptide of Met-12
  MOD_RES (1)..(12): Desamino modification
  MOD_RES (12)..(12): AMIDATION
YIYNVAGLYIHH

SEQ ID NO: 5 - protein, 12 aa, Artificial Sequence
  Artificial derivative of C-terminal amide peptide of Met-12
  MOD_RES (1)..(12): Desamino modification
  MOD_RES (12)..(12): AMIDATION
YVYNVAGLYVHH

SEQ ID NO: 6 - protein, 12 aa, Artificial Sequence
  Artificial derivative of C-terminal amide peptide of Met-12
  MOD_RES (1)..(12): Desamino modification
  MOD_RES (12)..(12): AMIDATION
YVYNVAGLYVHH

SEQ ID NO: 7 - protein, 12 aa, Artificial Sequence
  Artificial derivative of Met-12
  MOD_RES (1)..(12): Hydroxy-desamino modification
  MOD_RES (2)..(2): aIle
  MOD_RES (10)..(10): aIle
YIYNVAGLYIHH

SEQ ID NO: 8 - protein, 12 aa, Artificial Sequence
  Artificial derivative of C-terminal amide peptide of Met-12
  MOD_RES (1)..(12): Desamino modification
  MOD_RES (12)..(12): AMIDATION (piperazine amide)
YVYNVAGLYVHH

(19) United States
(12) Patent Application Publication
(10) Pub. No.: US 2021/0260168 A1
(43) Pub. Date: Aug. 26, 2021

(54) COMPOSITIONS AND METHODS OF FAS INHIBITION

(71) Applicants: ONL Therapeutics, Inc., Ann Arbor, MI (US); The Schepens Eye Research Institute, Inc., Boston, MA (US)

(72) Inventors: David ZACKS, Ann Arbor, MI (US); Andrew KOCAB, Farmington Hills, MI (US); John FRESHLEY, Ann Arbor, MI (US); Meredith GREGORY-KSANDER, Boston, MA (US); Anitha KRISHNAN, Braintree, MA (US); Jana VAN DE GOOR, Ann Arbor, MI (US); Alexander BRIDGES, Saline, MI (US)

(21) Appl. No.: 17/026,005

(22) Filed: Sep. 18, 2020

Related U.S. Application Data
(63) Continuation of application No. PCT/US2019/023207, filed on Mar. 20, 2019.
(60) Provisional application No. 62/645,769, filed on Mar. 20, 2018.
(60) Provisional application No. 62/700,097, filed on Jul. 18, 2018.

(51) Int. Cl. A61K 38/45; A61K 48/00; C12N 9/12

(52) U.S. Cl. CPC A61K 38/45; C12N 9/1205; A61K 48/005

(57) ABSTRACT

Described are compositions and methods for preventing, treating or ameliorating an inflammation-mediated and/or complement-mediated disease or condition in a subject comprising administering to the subject a Fas inhibitor, its derivative, a pharmaceutically acceptable salt thereof, of a gene therapy encoding the Fas inhibitor in an amount effective to inhibit Fas signaling.

### RELATED APPLICATIONS

This application is a continuation of US International Application PCT/US2019/023207 filed Mar. 20, 2019, which claims the benefit of the filing date under 35 U.S.C. § 119(e) of Provisional U.S. Patent Application Ser. Nos. 62/645,769, filed Mar. 20, 2018 and 62/700,097, filed Jul. 18, 2018, which are hereby incorporated by reference.

### SEQUENCE LISTING

The instant application contains a Sequence Listing which has been filed electronically in ASCII format and is hereby incorporated by reference in its entirety. Said ASCII copy, created on Mar. 20, 2019, is named 58109-702_301_SL.txt and is 3,520 bytes in size.

### BACKGROUND

Fas (CD95/APO-1) and its specific ligand (FASL/CD95L) are members of the tumor necrosis factor (TNF) receptor (TNF-R) and TNF families of proteins, respectively.

Interaction between Fas and FASL triggers a cascade of subcellular events that results in a definable cell death process in Fas-expressing targets. Fas is a 45 kDa type I membrane protein expressed constitutively in various tissues, including spleen, lymph nodes, liver, lung, kidney and ovary. (Leithauser, F. et al, Lab Invest, 69:415-429 (1993); Watanabe-Fukunaga, R. et al, J Immunol, 148:1274-1279 (1992)). FASL is a 40 kDa type II membrane protein, and its expression is predominantly restricted to lymphoid organs and perhaps certain immune-privileged tissues. (Suda, T. et al, Cell, 75:1169-1178 (1993); Suda, T. et al, J Immunol, 154:3806-3813 (1995)). In humans, FASL can induce cytolysis of FAS-expressing cells, either as a membrane-bound form or as a 17 kDa soluble form, which is released through metalloproteinase-mediated proteolytic shedding. (Kayagaki, N. et al, J Exp Med, 182:1777-1783 (1995); Mariani, S. M. et al, Eur J Immunol, 25:2303-2307 (1995)).

Binding of Fas ligands (FasL) to Fas receptor can elicit apoptotic signals either via classical pathways or via indirect pathways (Mundle & Raza., Trends. Immuno., 23:187-194 (2002)). Independently, Fas and FasL stimulation alone can induce cell proliferation (Aggarwal et al., FEBS Lett, 364:5-8 (1995); Freiberg et al, J Invest Dermatol, 108:215-219 (1997); Jelaska & Korn, J. Cell. Physiol, 175:19-29 (1998); Suzuki et al, J Immunol, 165:5537-5543 (2000); Suzuki et al, J. Exp. Med., 187: 123-8 (1998)). Membrane bound TNF superfamily members including FasL has been show to “reverse-signal” via their membrane attach cytoplasmic tail and thus they also possess a “bi-directional” signaling (Sun & Fink, J. Immuno., 179:4307-4312 (2007)). These studies suggest that small molecules, such as Kp 7 and mimetics thereof, which bind to both Fas and FasL can regulate Fas receptor signaling in a tissue-specific manner can be used to treat a variety of autoimmune pathologies.

The FASL/FAS system has been implicated in the control of the immune response and inflammation, the response to infection, neoplasia, and death of parenchymal cells in several organs. (Nagata et al supra; Biancone, L. et al., J Exp Med, 186:147-152 (1997); Krammer, P. H. Adv Immunol, 71 :163-210 (1999); Seino, K. et al, J Immunol, 161 :4484-4488 (1998)). Defects of the FASL/FAS system can limit lymphocyte apoptosis and lead to lymphoproliferation and autoimmunity. A role for FASL-FAS in the pathogenesis of rheumatoid arthritis, Sjogren's syndrome, multiple sclerosis, viral hepatitis, renal injury, inflammation, aging, graft rejection, HIV infection and a host of other diseases has been proposed. (Famularo, G., et al., Med. Hypotheses, 53:50-62 (1999)). FAS mediated apoptosis is an important component of tissue specific organ damage, such as liver injury that has been shown to be induced through the engagement of the FAS-FASL receptor system. (Kakinuma, C. et al., Toxicol Pathol, 27: 412-420 (1999); Famularo, G., et al., Med Hypotheses, 53: 50-62 (1999); Martinez, O. M. et al., Int Rev Immunol, 18:527-546 (1999); Kataoka, Y. et al, Immunology, 103:310-318 (2001); Chung, CS. et al, Surgery, 130:339-345 (2001); Doughty, L. et al, Pediatr Res, 52:922-927 (2002)).

Glaucoma is an eye disorder characterized by increased pressure inside the eye (“intraocular pressure” or “IOP”), excavation of the optic nerve head and gradual loss of the visual field. An abnormally high IOP is commonly known to be detrimental to the eye, and there are clear indications that, in glaucoma patients, this probably is the most important factor causing degenerative changes in the retina. The pathophysiological mechanism of open angle glaucoma is, however, still unknown. Unless treated successfully glaucoma will lead to blindness sooner or later, its course towards that stage is typically slow with progressive loss of the vision. IOP is the fluid pressure inside the eye. Tonometry is the method eye care professionals use to determine this. IOP is an important aspect in the evaluation of patients at risk of glaucoma. Most tonometers are calibrated to measure pressure in millimeters of mercury (mmHg).

In retinal cells, Fas receptor is activated by Fas ligand (FasL). Fas mediates cell death directly via multiple pathways: extrinsic apoptosis (through caspase cascade), intrinsic apoptosis (through Bid/Bax), and necroptosis (through RIPK1/3). Fas also mediates cell death indirectly through multiple immune response pathways: inflammasome (NLRP3, IL1β, TNFα), inflammasome-independent IL1β activation, HMGB1 nuclear release and secretion, and others yet to be determined.

Consequently, the FASL-FAS pathway represents an important general target for therapeutic intervention.

As such, there still exists a need for developing Fas inhibitors, compositions including Fas inhibitors, and methods of using the Fas inhibitors in order to prevent or ameliorate various diseases or conditions.

### SUMMARY

One embodiment relates to a method for preventing, treating or ameliorating an inflammation-mediated and/or complement-mediated disease or condition in a subject comprising administering to the subject a Fas inhibitor, its derivative, a pharmaceutically acceptable salt thereof, or a gene therapy encoding the Fas inhibitor in an amount effective to inhibit Fas signaling, wherein the inhibition of Fas signaling results in at least one (or at least two, or at least three, or at least four, etc., or all) of the following: reduction of expression or concentration of at least one Fas-mediated inflammation-related gene or protein (e.g. TNFα, IL-1β, IP-10, IL-18, MIP1α, IL-6, GFAP, MIP2, MCP-1, or MIP-1β); reduction of expression or concentration of at least one Fas-mediated complement-related gene or protein (e.g., complement component 3 (C3) and complement component 1q (C1q)); reduction of gene or protein expression or concentration of Caspase 8; reduction of gene or protein expression or concentration of one or more components of the inflammasome (e.g., NLRP3 and NLRP2); reduction of gene or protein expression or concentration of one or more C—X—C motif chemokines (e.g., CXCL2 (MIP-2α) and CXCL10 (IP-10)); reduction of gene or protein expression or concentration of one or more C—X3-C motif chemokines (e.g., CX3CL1 (fractalkine)); reduction of gene or protein expression or concentration of one or more C—C motif chemokines (e.g., CCL2 (MCP-1), CCL3 (MIP-1α), and CCL4 (MIP-1β)); reduction of gene or protein expression or concentration of toll-like receptor 4 (TLR4); reduction of gene or protein expression or concentration of one or more interleukin cytokines (e.g., IL-1β, IL-18, and IL-6); reduction of gene or protein expression or concentration of one or more TNF superfamily cytokines (e.g., TNFα); reduction of Fas-mediated Muller cell activation as indicated by reduced GFAP gene or protein expression or concentration; or increase of expression or concentration or prevent the reduction of expression or concentration of at least one pro-survival gene or protein, thereby preventing, treating, or ameliorating the disease or condition in the subject. The Fas inhibitor may be selected from the group consisting of: Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor. The subject may have or is at risk of having the inflammation-mediated and/or complement-mediated disease or condition. The inflammation-mediated and/or complement-mediated disease or condition may be retinal disease (e.g., glaucoma, retinal detachment, AMD (dry and wet), diabetic retinopathy, Uveitis, retinal vein occlusion, inherited retinal degenerations, including retinitis pigmentosa, or NAION), immunological disease, cancer, amyloid disease (e.g., Alzheimer's disease, type-2 diabetes, Huntington's disease, ALS, or Parkinson's disease), an injury caused by ischemia or reperfusion (e.g., stroke), autoimmune disease (e.g., allergy, lupus, or rheumatoid arthritis), neurodegeneration, and diseases of the central nervous system (e.g., neuropathy or a demyelinating disease selected from the group consisting of multiple sclerosis and inflammatory demyelinating diseases). The Fas inhibitor, its derivative, fragment, the gene therapy product, its corresponding interfering RNA (RNAi), or the pharmaceutically acceptable salt thereof may be administered in a pharmaceutical composition comprising the Fas inhibitor, its derivative, fragment, pharmaceutically acceptable salt, or a gene therapy that encodes the Fas inhibitor; and a pharmaceutically acceptable additive, such as carriers, excipients, disintegrators or disintegrating aids, binders, lubricants, coating agents, pigments, diluents, bases, dissolving agents or solubilizers, isotonic agents, pH regulators, stabilizers, propellants, and adhesives. In the method, the Fas inhibitor, its derivative, or the pharmaceutically acceptable salt thereof may be administered via an injection.

Yet another embodiment related to a method for preventing, treating or ameliorating an inflammation-mediated and/or complement-mediated disease or condition in a subject comprising administering to the subject a Fas inhibitor selected from the group consisting of Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor, in an amount effective to inhibit Fas signaling, and thereby prevent, treat or ameliorate the inflammation-mediated and/or complement-mediated disease or condition in the subject. The subject has or is at risk of having the inflammation-mediated and/or complement-mediated disease or condition. The inflammation-mediated and/or complement-mediated disease or condition may be retinal disease (e.g., glaucoma, retinal detachment, AMD (dry and wet), diabetic retinopathy, Uveitis, retinal vein occlusion, inherited retinal degenerations, including retinitis pigmentosa, or NAION), immunological disease, cancer, amyloid disease (e.g., Alzheimer's disease, type-2 diabetes, Huntington's disease, ALS, or Parkinson's disease), an injury caused by ischemia or reperfusion (e.g., stroke), autoimmune disease (e.g., allergy, lupus, or rheumatoid arthritis), neurodegeneration, and diseases of the central nervous system (e.g., neuropathy or a demyelinating disease selected from the group consisting of multiple sclerosis and inflammatory demyelinating diseases). The Fas inhibitor may be administered in a pharmaceutical composition comprising the Fas inhibitor and a pharmaceutically acceptable additive selected from the group consisting of carriers, excipients, disintegrators or disintegrating aids, binders, lubricants, coating agents, pigments, diluents, bases, dissolving agents or solubilizers, isotonic agents, pH regulators, stabilizers, propellants, and adhesives. The Fas inhibitor may be administered via an injection (e.g., an intravitreal injection, intrathecal, intravenous, or periocular injection).

Another embodiment related to a method for preserving retinal ganglion cells and axon density, or preventing the loss of ganglion cells and axon density in a patient with glaucoma comprising administering to the subject a Fas inhibitor, a derivative thereof, a fragment thereof, a pharmaceutically acceptable salt thereof, or a gene therapy encoding the Fas inhibitor, wherein the preserving or preventing the loss of retinal ganglion cells and axon density, or preventing the loss thereof is due to at least one (or at least two, or all three) of the following: inhibition of microglial/macrophage activation or recruitment; inhibition of at least one of TNF-α, CCL2/MCP-1 or CCL3/MIP-la gene or protein expression or concentration; or reduction of IL-1β gene or protein expression or protein maturation, wherein the Fas inhibitor is administered to the subject in an amount effective to inhibit Fas signaling. The Fas inhibitor, a derivative thereof, a fragment thereof, a pharmaceutically acceptable salt thereof, or a gene therapy encoding the Fas inhibitor may be administered in a pharmaceutical composition comprising the Fas inhibitor, a derivative thereof, a fragment thereof, a pharmaceutically acceptable salt thereof, or a gene therapy encoding the Fas inhibitor; and a pharmaceutically acceptable additive. The additive may be selected from the group consisting of carriers, excipients, disintegrators or disintegrating aids, binders, lubricants, coating agents, pigments, diluents, bases, dissolving agents or solubilizers, isotonic agents, pH regulators, stabilizers, propellants and adhesives. The composition may be in a form selected from the group consisting of: solution, pill, ointment, suspension, eye drops, gel, cream, foam, spray, liniment, and powder. The administering may be via an injection, wherein the injection is an intravitreal injection, intrathecal, intravenous or periocular injection. The composition may further comprise at least one non-ionic surfactant selected from the group consisting of Polysorbate 80, Polysorbate 20, Poloxamer 407, and Tyloxapol. The Fas inhibitor or the composition comprising the Fas inhibitor may be administered daily, twice daily, every other day, weekly, biweekly, monthly, bimonthly, or tri-monthly. The Fas inhibitor or the composition comprising Fas inhibitor may be administered in a daily dose of from about 1 ng to about 1 mg. The composition may be in the form of eye drops and the Fas inhibitor is in a concentration between 0.000001% w/v and 2% w/v.

Yet another embodiment relates to a method of treating a subject having at least a 10% increase in the mRNA and/or protein expression level(s) of at least one (or at least two, or at least three, or at least four, etc., or all) of the following gene and/or protein in the subject's eye, as compared to a control: at least one Fas-mediated inflammation-related gene or protein (e.g. TNFα, IL-1β, IP-10, IL-18, MIP1α, IL-6, GFAP, MIP2, MCP-1, or MIP-1β); at least one Fas-mediated complement-related gene or protein (complement component 3 (C3) or complement component 1q (C1q)); Caspase 8; one or more components of the inflammasome (e.g., NLRP3 or NLRP2); one or more C—X—C motif chemokines (e.g., CXCL2 (MIP-2α) or CXCL10 (IP-10)); one or more C—X3-C motif chemokines (e.g., CX3CL1 (fractalkine)); one or more C—C motif chemokines (CCL2 (MCP-1), CCL3 (MIP-1α), and CCL4 (MIP-1β)); toll-like receptor 4 (TLR4); one or more interleukin cytokines (e.g., IL-1β, IL-18, and IL-6); one or more TNF superfamily cytokines (e.g., TNFα); or GFAP gene or protein expression or concentration, the method comprising administering to the subject a Fas inhibitor. The Fas inhibitor may be any Fas inhibitor described herein. For example, the Fas inhibitor may be selected from the group consisting of: Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor.

Yet further embodiment relates to a method of treating a subject having at least a 5% increase in the mRNA and/or protein expression level(s) of at least one (or at least two, or at least three, or at least four, etc., or all) of the following gene and/or protein in the subject's serum, plasma, whole blood, or cerebrospinal fluid, as compared to a control: at least one Fas-mediated inflammation-related gene or protein (e.g. TNFα, IL-1β, IP-10, IL-18, MIP1α, IL-6, GFAP, MIP2, MCP-1, or MIP-1β; at least one Fas-mediated complement-related gene or protein (complement component 3 (C3) or complement component 1q (C1q)); Caspase 8; one or more components of the inflammasome (e.g., NLRP3 or NLRP2); one or more C—X—C motif chemokines (e.g., CXCL2 (MIP-2α) or CXCL10 (IP-10)); one or more C—X3-C motif chemokines (e.g., CX3CL1 (fractalkine)); one or more C—C motif chemokines (CCL2 (MCP-1), CCL3 (MIP-1α), and CCL4 (MIP-1β)); toll-like receptor 4 (TLR4); one or more interleukin cytokines (e.g., IL-1β, IL-18, and IL-6); one or more TNF superfamily cytokines (e.g., TNFα); or GFAP gene or protein expression or concentration, the method comprising administering to the subject a Fas inhibitor, the method comprising administering to the subject a Fas inhibitor. The Fas inhibitor may be any Fas inhibitor described herein. For example, the Fas inhibitor may be selected from the group consisting of: Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor.

Yet, a further embodiment relates to a composition comprising a compound selected from the group consisting of Compounds 2-8, a derivative thereof, an analog thereof, or a fragment thereof.

### DESCRIPTION OF THE DRAWINGS

FIG. 1 depicts bar graphs showing the expression of the inflammation-related genes: (A) TNF, (B) IL-1β, (C) IP-10, (D) IL-18, (E) MIP-1α, (F) IL-6, (G) GFAP, (H) MIP2, and (i) Complement C3 in samples treated with Compound 1, as compared to the vehicle and microbeads alone.

FIG. 2 depicts bar graphs showing the expression of genes: (A) MCP-1, (B) Caspase 8, (C) CFLIP, (D) TLR-4, (E) MIP-1β, (F) NLRP3, and (G) Complement C1Q in samples treated with Compound 1, as compared to the vehicle and microbeads alone.

FIG. 3 depicts bar graphs showing the expression of genes: (A) Bax, (B) FADD, (C) ASC, (D) FasR, (E) FasL, (F) Complement C4, (G) NLRP2, and (H) Caspase 3 in samples treated with Compound 1, as compared to the vehicle and microbeads alone.

FIG. 4 depicts IOP graph for the study with drug/vehicle given at the same time as microbeads.

FIG. 5 depicts IOP graph for the study with drug/vehicle injection 7 days post-injection of microbeads.

FIG. 6 depicts representative images from RGC and axon counts for drug/vehicle injected at the same time as microbeads/saline.

FIG. 7 depicts a bar graph based on the quantification of the collected images for RGC cell density.

FIG. 8 depicts a bar graphs based on the quantification of the collected images for axon density.

FIG. 9 depicts representative images from RGC and axon data for the day 7 drug/vehicle injection study.

FIG. 10 depicts a bar graph based on the quantification of the collected images for RGC cell density for day 7 drug/vehicle injection study.

FIG. 11 depicts a bar graph based on the quantification of the collected images for axon density for day 7 drug/vehicle injection study.

FIG. 12 depicts images showing that treatment with Compound 1 inhibits the activation of retinal microglia and/or the infiltration of macrophages into the retina following elevated IOP, and the quantification of process length of the microglia (bar graph).

FIG. 13 depicts a bar graph for Western blot analysis following microbead injection in the mice treated with Compound 1 as compared to vehicle.

### DETAILED DESCRIPTION

Provided herein are Fas inhibitors, compositions thereof, pharmaceutical preparations thereof, as well as therapeutic methods.

PCT Pub. No. WO 2016/178993A1, U.S. Non-provisional application Ser. No. 15/570,948, filed on Oct. 31, 2017, and U.S. Provisional U.S. Patent Application Ser. No. 62/155,711, filed May 1, 2015, are hereby incorporated by reference in their entirety in order to more fully describe the state of the art as known to those skilled therein as of the date of the invention described and claimed herein.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, compositions, devices and materials are described herein.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The terms “optional” or “optionally” mean that the subsequently described event, circumstance, or component may but need not occur, and that the description includes instances where the event or circumstance occurs and instances in which it does not.

As used herein, the term “about” modifying, for example, the quantity of an ingredient in a composition, concentration, volume, process temperature, process time, yield, flow rate, pressure, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities. Further, where “about” is employed to describe a range of values, for example “about 1 to 5” the recitation means “1 to 5” and “about 1 to about 5” and “1 to about 5” and “about 1 to 5,” unless specifically limited by context.

As used herein, “treatment” refers to a clinical intervention made in response to a disease, disorder or physiological condition manifested by a patient or to which a patient may be susceptible. The aim of treatment includes, but is not limited to, the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Subjects in need of treatment include those already affected by a disease or disorder or undesired physiological condition as well as those in which the disease or disorder, or undesired physiological condition is to be prevented. In certain embodiments, treatment refers to the alleviation or prevention of symptoms, slowing or stopping the progression or worsening of an inflammation-mediated and/or complement-mediated pathology and/or tissue damage in a disease, disorder, or condition to be treated with Fas inhibitors, as described in detail below, and/or the remission of the disease, disorder or condition.

The term “express” and “expression” means allowing or causing the information in a gene or DNA sequence to become manifest, for example producing RNA (such as rRNA or mRNA) or a protein by activating the cellular functions involved in transcription and translation of a corresponding gene or DNA sequence. The term “reduction of expression or concentration” refers to a decrease in production or amount of the specified gene or protein. The term “gene,” means a DNA sequence that codes for or corresponds to a particular sequence of amino acids, which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription.

As used herein, a “subject” or “patient” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human.

A “control” is an alternative subject or sample used in an experiment for comparison purposes. A control can be “positive” or “negative.” For example, where the purpose of the experiment or the comparison in a method is to determine a correlation of an patient treatment with a particular symptom, one may use either a positive control (a patient exhibiting the symptom and not subjected to the treatment, or a sample from such a patient), and/or a negative control (a subject that does not exhibit the symptom and not subjected to the treatment, or a sample from such a subject).

The term “reduced” or “reduce” as used herein generally means a decrease by at least 5% as compared to a reference or control level, for example, a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease, or any integer decrease between 10-100% as compared to a reference or control level.

The term “increased” or “increase” as used herein generally means an increase of at least 5% as compared to a reference or control level, for example an increase of at least 10% as compared to a reference level, or at least about 20%, or at least about 30%, or at least about 40%, or at least about 50%, or at least about 60%, or at least about 70%, or at least about 80%, or at least about 90% or up to and including a 100% increase or any integer increase between 10-100% as compared to a reference level, or about a 2-fold, or about a 3-fold, or about a 4-fold, or about a 5-fold or about a 10-fold increase, or any increase between 2-fold and 10-fold or greater as compared to a reference or control level.

### Fas Inhibitors

Certain embodiments relate to Fas inhibitors and their use in methods of inhibiting Fas activation and/or signaling leading to preventing, treating, or ameliorating various diseases or conditions. Importantly, by inhibiting Fas activation and/or signaling, inflammation-mediated and/or complement-mediated diseases or conditions may be prevented, treated and/or ameliorated.

As used herein the term “Fas inhibitor” refers to a compound capable of inhibiting or reducing Fas receptor activation and/or signaling either via classical pathways or via indirect pathways. Fas inhibitor may bind to the Fas receptor and directly or indirectly affect the gene and protein expression or activity of molecules downstream of the Fas pathway, to prevent inflammation-mediated and/or complement-mediated diseases or conditions. Fas inhibitors are described in detail below and include any derivatives, fragments, and pharmaceutically acceptable salts of the described Fas inhibitors. As used herein, the term “pharmaceutically acceptable salt” refers to any acid or base of a pharmaceutical agent or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts. Fas inhibitors may also include gene therapy agents. For example, Fas inhibitors may include polynucleotides (e.g., Fas polynucleotide antagonists, such as short interfering RNAs (siRNA) or clustered regularly interspaced short palindromic repeat RNAs (CRISPR-RNA or crRNA, including single guide RNAs (sgRNAs) having a crRNA and tracrRNA sequence, as described in more detail below.

The term “Fas-mediated” means involving or depending on the Fas receptor and/or its activation.

Exemplary Fas inhibitors for use in the described methods are provided below.

In certain embodiments, Fas inhibitors for use in the described methods include any Met and Met-derived peptides and/or fragments. The Met protein has been described previously in U.S. Pat. Pub Nos. US 2007/0184522 and US 2008/0280834, and by Wang et al., Molecular Cell, 9:411-421 (2002) and Zou et al., Nature Medicine, 13(9):1078-1085 (2007), which are incorporated by reference in their entirety. The Met protein, also called c-Met or hepatocyte growth factor receptor (HGF receptor), is encoded by the Met gene. Met is comprised of two major subunits: the a and _{R} subunits. Met and fragments of Met, including the extracellular domain of Met and its a subunit, have been shown to bind to Fas and prevent cells from undergoing apoptosis (Wang et al., Molecular Cell, 9:411-421 (2002)). The Met-Fas interaction is thought to sequester Fas and prevent its trimerization, thereby preventing FasL trimers from binding a trimerized receptor complex. Certain Met-derived peptides, include Met-12, have been shown to have similar effects, leading to Fas inhibition to promote cell survival (Zou et al., Nature Medicine, 13(9):1078-1085 (2007)).

Another example of Fas inhibitor is Met-12 (Met-12 has been previously described in U.S. Pat. No. 8,343,931, which is incorporated herein in its entirety), a derivative, and a pharmaceutically active salt thereof.

A further example of Fas inhibitor includes Compound 1 of Formula 1, which is a C-terminal amide peptide of Met-12, a derivative, and a pharmaceutically active salt thereof:

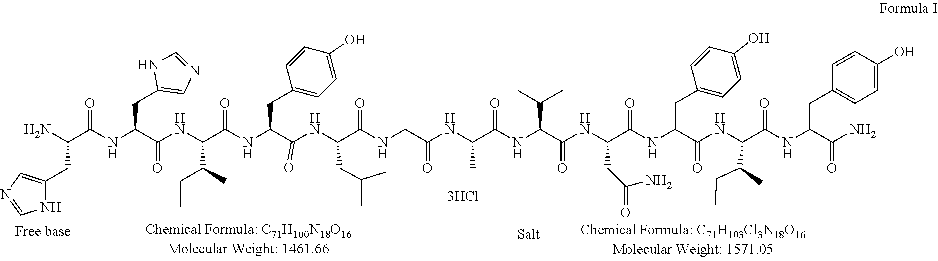 [SMILES: CC[C@H](C)[C@H](NC(=O)C(Cc1ccc(O)cc1)NC(=O)[C@H](CC(N)=O)NC(=O)[C@@H](NC(=O)[C@H](C)NC(=O)CNC(=O)[C@H](CC(C)C)NC(=O)[C@H](Cc1ccc(O)cc1)NC(=O)[C@@H](NC(=O)[C@H](Cc1cnc[nH]1)NC(=O)[C@@H](N)Cc1cnc[nH]1)[C@@H](C)CC)C(C)C)C(=O)NC(Cc1ccc(O)cc1)C(N)=O]

Compound 1/Formula I: His-His-Ile-Tyr-Leu-Gly-Ala-Val-Asn-Tyr-Ile-Tyr-amide (SEQ ID NO:1)

Other examples of Fas inhibitors include derivatives or analogs, and pharmaceutically acceptable salts of Met-12 peptide or Compound 1, including Compounds II-VIII below:

Formula II/Compound 2:

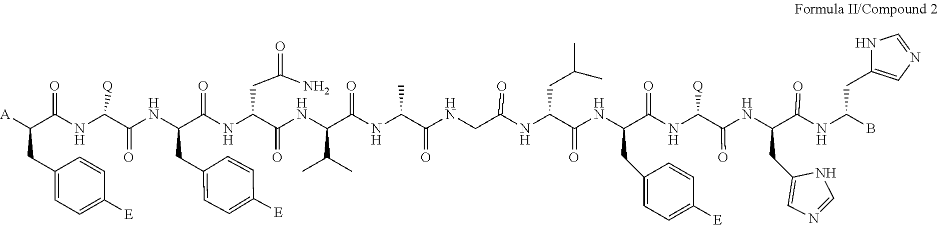

Formula II/ Compound 2: H-D-Tyr-D-Ile-D-Tyr-D-Asn-D-Val-D-Ala-Gly-D-Leu-D-Tyr-D-Ile-D-His-D-His-NH2 (SEQ ID NO: 2).

wherein:

A is H—, OH—, NH₂—, G¹(CH₂)ₙ—, R¹CONH—, or R²O—;

B is —H, CH₂OH, CH₂OR², —CHO, —CO₂R², —CONH₂, —CONHR², —CONR³₂, —CONH(CH₂)_{y}NR³₂, —(CH₂)ₙ-G¹, —COCH₂-G¹, —CONHCH₂-G¹, —(CH₂)ₙNH₂, —(CH₂)ₙNHR², —(CH₂)ₙNR³₂, NH-[D]Glu-[D]-His-OH, NH-[D]Glu-[D]-His-NH², -[D]Ala-[D]-His-NH₂, -Gly-[D]-His-NH₂, or CONH(CH₂)ₙ-G²;

E, at each occurrence, is independently —H, —OH, OR⁴, SH, SR⁴, or halogen;

G¹, at each occurrence, is independently —H, —C(═O)NH², —C(═O)NHR², —C(═O)NR³₂, C(═O)OR², or —C(═O)R¹;

G² at each occurrence is a heteroalicyclic ring of 4-7 members comprising at least one tertiary amine functionality NR² within the ring, or an alicyclic ring of 3-7 members substituted with NR³₂;

L, at each occurrence, is a multivalent polyethylene glycol derivative with 2-4 termini, each of which may be independently capped with H, R⁵ or another molecule of the peptide of Formula I;

Q, at each occurrence, is independently, [R]-I-methylethyl, [S]-I-methylethyl, 2-propyl, 2-methyl-prop-2-yl, C₃₋₆-cycloalkyl, C₄₋₆-cycloalkenyl, [R]- or [5]-tetrahydrofuran-2-yl, [R]- or [5]-tetrahydrofuran-3-yl, [R]- or [5]-tetrahydrothienyl-2-yl, [R]- or [5]-tetrahydrothienyl-3-yl, [R]- or [S]-tetrahydropyran-2-yl, [R]- or [S]-tetrahydropyran-3-yl, [R]- or [S]-tetrahydropyran-4-yl, [R]- or [5]-tetrahydrothiopyran-2-yl, [R]- or [S]-tetrahydrothiopyran-3-yl, tetrahydrothiopyran-4-yl or [R]- or [S]-I-(R⁵O)ethyl;

R¹, at each occurrence, is independently H, C₁₋₆alkyl, —(CH₂)ₓ(OCH₂CH₂)ₘOR⁵, C₁₋₆ alkoxy or L;

R², at each occurrence, is independently C₁₋₆alkyl, C₂₋₆alkyl substituted with OR⁵ or NR⁵₂, —(CH₂)ₓ(OCH₂CH₂)ₘOR⁵ or L;

R³, at each occurrence, is independently C₁₋₆alkyl, C₂₋₆alkyl substituted with OR⁵ or NR⁵₂, —(CH₂)ₓ(OCH₂CH₂)ₘOR⁵;

or two R³s, taken together with the N atom to which they are attached, may form a monocyclic ring of 4-8 members or a fused, bridged or spiro bicyclic ring of 6-10 members, which can include up to two groups within the ring chosen independently from —O—, —(C═O)—, NR⁶, S, SO, or SO₂;

R⁴, at each occurrence, is independently C₁₋₆alkyl, C₁₋₆acyl, or —OPO₃R⁵₂;

R⁵, at each occurrence, is independently H or C₁₋₆alkyl;

R⁶, at each occurrence, is H, C₁₋₆alkyl, C₂₋₆hydroxyalkyl, C₁₋₆alkoxy-, C₁₋₆alkyl, or C₁₋₆acyl;

m=1-100;

n=0-3;

x=0-6; and

y=2-4, and

wherein at most one of R¹ and R² is L.

Formula III/Compound 3:

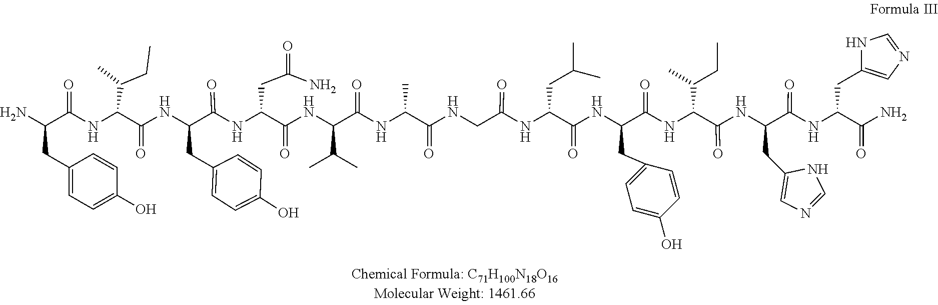 [SMILES: CC[C@@H](C)[C@@H](NC(=O)[C@H](N)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)[C@H](C)CC)C(C)C]

Compound 3/Formula 3: All [D]Tyr-Ile-Tyr-Asn-Val-Ala-Gly-Leu-Tyr-Ile-His-His-amide (SEQ ID NO:3)

Formula IV/Compound 4:

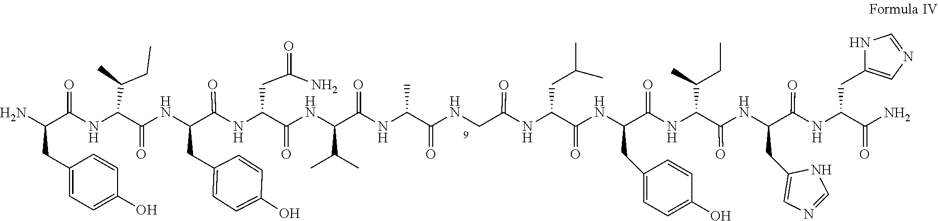 [SMILES: CC[C@H](C)[C@@H](NC(=O)[C@H](N)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)[C@@H](C)CC)C(C)C]

Compound 4/Formula IV: All [D]Tyr-allo-Ile-Tyr-Asn-Val-Ala-Gly-Leu-Tyr-allo-Ile-His-His-amide (SEQ ID NO:4)

Formula V/Compound 5:

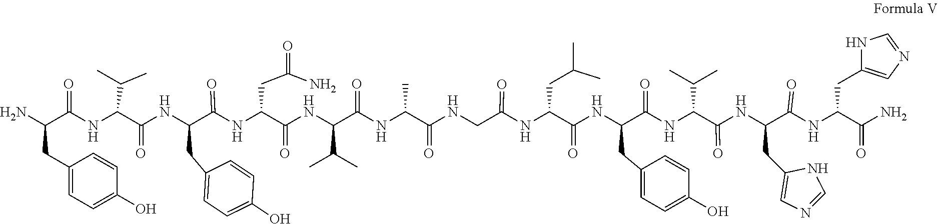 [SMILES: CC(C)C[C@@H](NC(=O)CNC(=O)[C@@H](C)NC(=O)[C@H](NC(=O)[C@@H](CC(N)=O)NC(=O)[C@@H](Cc1ccc(O)cc1)NC(=O)[C@H](NC(=O)[C@H](N)Cc1ccc(O)cc1)C(C)C)C(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)C(C)C]

Compound 4/Formula IV: All [D]Tyr-Val-Tyr-Asn-Val-Ala-Gly-Leu-Tyr-Val-His-His-amide (SEQ ID NO:5)

Formula VI/Compound 6:

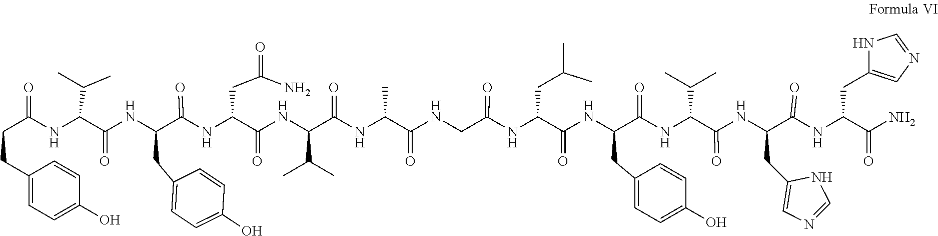

Compound 6/Formula VI: All [D](DesaminoTyr)-Val-Tyr-Asn-Val-Ala-Gly-Leu-Tyr-Val-His-His-amide (SEQ ID NO:6)

Formula VII/Compound 7:

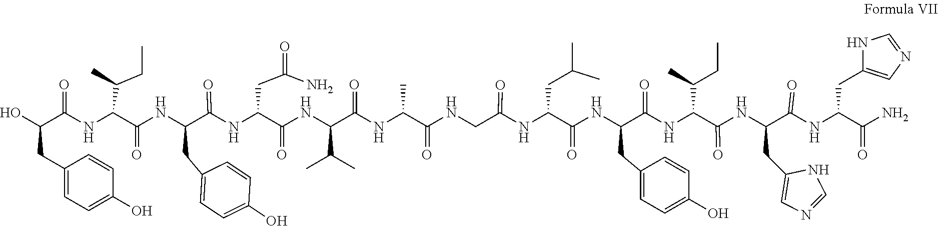 [SMILES: CC[C@H](C)[C@@H](NC(=O)[C@H](O)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)[C@@H](C)CC)C(C)C]

Compound 7/Formula VII: All [D](Hydroxy-desaminoTyr)-allo-Ile-Tyr-Asn-Val-Ala-Gly-Leu-Tyr-allo-Ile-His-Histamine (SEQ ID NO:7)

Formula VIII/Compound 8:

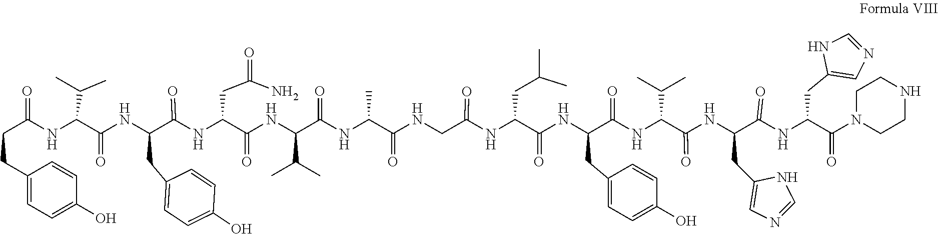

Compound 8/Formula VIII: All [D](DesaminoTyr)-Val-Tyr-Asn-Val-Ala-Gly-Leu-Tyr-Val-His-His-piperazine amide (SEQ ID NO:8)

In some embodiments, a Fas inhibitor may be a polypeptide comprising any of Compounds I-VIII and can be prepared by methods known to those of ordinary skill in the art. For example, a peptide can be synthesized using solid phase polypeptide synthesis techniques (e.g., Fmoc or tBoc) with D-amino acids. Alternatively, the polypeptide can be synthesized using solution phase techniques, using a wide variety of protected D-amino acids. For example, Compound 2 can be obtained by building the retro-inverso (R-I) Met-12 peptide sequence, (d)Y(d)1(d)Y(d)N(d)V(d)AG(d)L(d)Y(d)I(d)H(d)H (alternatively, “yiynvaglyihh,” using the convention of small letters for d-amino acids and noting that glycine is achiral) onto an amino resin, as is known to those of skill in the art to produce after deprotection and resin cleavage its C-terminal amide, (d)Y(d)1(d)Y(d)N(d)V(d)AG(d)L(d)Y(d)I(d)H(d)H-NH2, Compound 2 (SEQ ID NO:2).

Specifically, although Compound 2 can be obtained conceptually from the c-Met sequence by a normal hydrolysis between residues 59 and 60, and an unnatural breaking of the peptide chain between the peptide nitrogen and the a-carbon of residue 72, rather than at the carbonyl carbon of residue 71, and then reversing the entire sequence whilst exchanging the eleven chiral amino acid residues for their enantiomers, this is not something that could occur naturally, as neither the required bond break between residues 71 and 72, nor the retro-inverso c-Met protein occur in nature. This is not a cleavage, which occurs naturally.

In certain embodiments, analogs or derivatives of Met-12 or C terminal amide thereof can be produced by converting retro-inverso Met-12 into its C-terminal primary amide, to form Compound 2, although it is generally more practical to build up the peptide from an already aminated first amino acid residue, by use of an amino resin, familiar to one of skill in the art.

In certain embodiments, Compounds 1-8 or c-Met, c-Met protein fragments, c-Met polypeptides, and analogs or derivatives of these molecules, such as Met-12, may be linked with various other molecules (e.g. PEG, other active therapeutic molecules, various molecules commonly known as linkers) to optimize delivery, potency, and/or other pharmaceutical properties. These linkers may be covalent and permanent or designed to degrade or be processed over time.

In certain embodiments, c-Met, c-Met protein fragments, c-Met polypeptides, and analogs or derivatives of these molecules may be modified to include amino acids substitutions such as ones known to those skilled in the art including but not limited to substitutions to maintain or modify polarity or size, etc. or substitutions or sequences that contain non-proteinogenic amino acids or various terminal caps or modifications, each or multiple in combination which do not occur naturally.

In certain embodiments, Compounds 1-8 or c-Met protein fragments, c-Met polypeptides, and analogs or derivatives of these molecules, such as Met-12, could be mimicked through petidomimetic strategies by those skilled in the art.

Additional Fas inhibitors include Fas antibody inhibitors, Kp7-6, and viral vector-based gene therapy inhibitors of Fas, including viral vector constructs that lead to the production and/or secretion of Fas inhibiting proteins and viral vector constructs that lead to the production and/or secretion of small peptides like Met12 and analogs, including, e.g., c-MET, c-Met alpha subunit, c-Met alpha subunit modified to prevent binding of HGF.

In certain embodiments, described herein are methods for preventing, treating or ameliorating an inflammation-mediated and/or complement-mediated disease or condition in a subject that involve gene therapy. As used herein, the term “gene therapy” refers to the introduction of extra genetic material in the form of DNA or RNA into the total genetic material in a cell that restores, corrects, or modifies expression of a gene, or for the purpose of expressing a therapeutic polypeptide, e.g., a Fas inhibitor.

Specifically, methods for preventing, treating or ameliorating an inflammation-mediated and/or complement-mediated disease or condition in a subject that comprise administering to the subject a gene therapy encoding the Fas inhibitor in an amount effective to inhibit Fas signaling are described.

Gene therapy uses a gene therapy agent. As used herein, the term “a gene therapy agent” refers to any nucleic acid construct that encodes and results in the expression of a Fas inhibitor, which is capable of transforming a cell in or adjacent to the body lumen. Transformation refers to the process of changing the genotype of a recipient cell by the stable introduction of RNA or DNA by any methodology available to one of ordinary skill in the art. Any gene therapy agent that encodes and results in the expression of a Fas inhibitor may be used.

In order to express a desired polypeptide, e.g., Fas inhibitor, the introduction or delivery of DNA or RNA into cells can be accomplished by multiple methods using a vector (or a vector system), or a carrier. The two major classes of vector systems are recombinant viruses (also referred to as biological nanoparticles or viral vectors), and naked DNA or DNA complexes (non-viral methods, e.g., via a carrier). Both classes of vectors may be used to prepare the gene therapy agents for use in the described methods.

The nucleic acid construct may be an RNA or DNA construct. Examples of types of nucleic acid constructs which may be used as the gene therapy agent include, but are not limited to strands or duplexes of DNA and RNA, DNA and RNA viral vectors and plasmids.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Examples of vectors are plasmids (e.g., DNA plasmids or RNA plasmids), autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4N5-DEST™, pLenti6N5-DEST™, and pLenti6.2N5-GW/lacZ (Invitrogen) for lentivirus-mediated gene transfer and expression in mammalian cells. In certain embodiments, useful viral vectors include, e.g., replication defective retroviruses and lentiviruses.

The term “viral vector” may refer either to a virus (e.g., a transfer plasmid that includes virus-derived nucleic acid elements that typically facilitate transfer of the nucleic acid molecule or integration into the genome of a cell; e.g. virus-associated vector), or viral particle capable of transferring a nucleic acid construct into a cell, or to the transferred nucleic acid itself. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cell's genome. Viral vectors and transfer plasmids contain structural and/or functional genetic elements that are primarily derived from a virus. Exemplary viruses used as vectors include retroviruses, adenoviruses, adeno-associated viruses, lentiviruses, pox viruses, alphaviruses, and herpes viruses. For example, the term “retroviral vector” refers to a viral vector or plasmid containing structural and functional genetic elements, or portions thereof, that are primarily derived from a retrovirus; the term “lentiviral vector” refers to a viral vector or plasmid containing structural and functional genetic elements, or portions thereof, including LTRs that are primarily derived from a lentivirus. The term “hybrid vector” refers to a vector, LTR or other nucleic acid containing both retroviral, e.g., lentiviral, sequences and non-lentiviral viral sequences. In one embodiment, a hybrid vector refers to a vector or transfer plasmid comprising retroviral e.g., lentiviral, sequences for reverse transcription, replication, integration and/or packaging.

The term “construct,” as used herein, refers to a recombinant nucleic acid that has been generated for the purpose of the expression of a specific nucleotide sequence(s), or that is to be used in the construction of other recombinant nucleotide sequences.

The terms “polynucleotide,” or “nucleic acid” are interchangeable and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The “Fas inhibitor polynucleotide” includes polymers of nucleotides of any length, and include DNA and RNA for Fas inhibitors, including fragments thereof

The term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV) and lentivirus.

The term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis encephalitis virus (CAEV); equine infectious anemia virus (EIAV); feline immunodeficiency virus (FIV); bovine immune deficiency virus (BIV); and simian immunodeficiency virus (SIV).

The terms “lentiviral vector,” “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles of the disclosure and are present in DNA form in the DNA plasmids of the disclosure.

As used herein, the term “transfection” refers to the introduction of a nucleic acid into a host cell, such as by contacting the cell with a recombinant AAV virus as described below.

Adeno-associated virus (AAV) is a replication-deficient parvovirus, the single-stranded DNA genome of which is about 4.7 kb in length including 145 nucleotide inverted terminal repeat (ITRs). The ITRs play a role in integration of the AAV DNA into the host cell genome. When AAV infects a host cell, the viral genome integrates into the host's chromosome resulting in latent infection of the cell. In a natural system, a helper virus (for example, adenovirus or herpesvirus) provides genes that allow for production of AAV virus in the infected cell. In the case of adenovirus, genes E1A, E1B, E2A, E4 and VA provide helper functions. Upon infection with a helper virus, the AAV provirus is rescued and amplified, and both AAV and adenovirus are produced. In the instances of recombinant AAV vectors having no Rep and/or Cap genes, the AAV can be non-integrating. In some embodiments, the non-integrating AAV is preferably used to produce the

AAV vectors that comprise coding regions of one or more proteins of interest, for example proteins that are more than 500 amino acids in length, are provided. The AAV vector can include a 5′ inverted terminal repeat (ITR) of AAV, a 3′ AAV ITR, a promoter, and a restriction site downstream of the promoter to allow insertion of a polynucleotide encoding one or more proteins of interest, wherein the promoter and the restriction site are located downstream of the 5′ AAV ITR and upstream of the 3′ AAV ITR. In some embodiments, the recombinant AAV vector includes a posttranscriptional regulatory element downstream of the restriction site and upstream of the 3′ AAV ITR. In some embodiments, the AAV vectors disclosed herein can be used as AAV transfer vectors carrying a transgene encoding a protein of interest for producing recombinant AAV viruses that can express the protein of interest in a host cell.

Generation of the viral vector can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)).

For example, U.S. Pat. No. 9,527,904B2, which is incorporated herein by reference, describes methods for delivery of proteins of interest using adeno-associated virus (AAV) vectors.

In some embodiments, a cell may be transfected with a recombinant AAV virus, e.g. AAV2, including the Fas inhibitor nucleic acid construct to encode and express the Fas inhibitor. For example, AAV vector including Fas inhibitor polynucleotide may be introduced into a target cell, e.g., a Muller or photoreceptor cell. Fas inhibitor may be Met-12, its amide derivative, Compound 1, or any other Fas inhibitor described herein, including derivatives, fragments and salts thereof.

In certain other embodiments, the delivery of a gene(s) or other polynucleotide sequence using viral vectors may be by means of viral infection (“transduction”).

In particular embodiments, host cells transduced with viral vector of the disclosure that expresses one or more polypeptides, are administered to a subject to treat and/or prevent and/or ameliorate inflammation-mediated and/or complement-mediated diseases or conditions described herein

In some embodiments, a cell may be transduced with a retroviral vector, e.g., a lentiviral vector, encoding an engineered Fas inhibitor construct. The transduced cells elicit a stable, long-term, and persistent cell response.

At each end of the provirus are structures called “long terminal repeats” or “LTRs.” The term “long terminal repeat (LTR)” refers to domains of base pairs located at the ends of retroviral DNAs which, in their natural sequence context, are direct repeats and contain U3, Rand U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, Rand U5. The U3 region contains the enhancer and promoter elements. The U5 region is the sequence between the primer binding site and the R region and contains the polyadenylation sequence. The R (repeat) region is flanked by the U3 and U5 regions. The LTR composed of U3, Rand U5 regions and appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site).

As used herein, the term “packaging signal” or “packaging sequence” refers to sequences located within the retroviral genome, which are required for insertion of the viral RNA into the viral capsid or particle, see e.g., Clever et al., 1995. J of Virology, Vol. 69, No. 4; pp. 2101-2109. Several retroviral vectors use the minimal packaging signal (also referred to as the psi ['P] sequence) needed for encapsidation of the viral genome. Thus, as used herein, the terms “packaging sequence,” “packaging signal,” “psi” and the symbol “P,” are used in reference to the non-coding sequence required for encapsidation of retroviral RNA strands during viral particle formation.

In various embodiments, vectors may comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective. As used herein, the term “replication-defective” refers to virus that is not capable of complete, effective replication such that infective virions are not produced (e.g., replication-defective lentiviral progeny). The term “replication-competent” refers to wild-type virus or mutant virus that is capable of replication, such that viral replication of the virus is capable of producing infective virions (e.g., replication-competent lentiviral progeny).

“Self-inactivating” (SIN) vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. In a further embodiment, the 3′LTR is modified such that the U5 region is replaced, for example, with an ideal poly(A) sequence. It should be noted that modifications to the LTRs such as modifications to the 3′LTR, the 5′LTR, or both 3′ and 5′LTRs, are also contemplated herein.

An additional safety enhancement may be provided by replacing the U3 region of the 5′LTR with a heterologous promoter to drive transcription of the viral genome during production of viral particles. Examples of heterologous promoters which may be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In certain embodiments, the heterologous promoter has additional advantages in controlling the manner in which the viral genome is transcribed. For example, the heterologous promoter may be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors may comprise a TAR element. The term “TAR” refers to the “trans-activation response” genetic element located in the R region of lentiviral (e.g., HIV) LTRs. This element interacts with the lentiviral trans-activator (tat) genetic element to enhance viral replication.

The “R region” refers to the region within retroviral LTRs beginning at the start of the capping group (i.e., the start of transcription) and ending immediately prior to the start of the poly A tract. The R region is also defined as being flanked by the U3 and U5 regions. The R region plays a role during reverse transcription in permitting the transfer of nascent DNA from one end of the genome to the other.

The term “FLAP element” refers to a nucleic acid whose sequence includes the central polypurine tract and central termination sequences (cPPT and CTS) of a includes the central polypurine tract and central termination sequences (cPPT and CTS) of a retrovirus, e.g., HIV-I or HIV-2. Suitable FLAP elements are described in U.S. Pat. No. 6,682,907 and in Zennou, et al., 2000, Cell, IO 1: 173. During HIV-I reverse transcription, central initiation of the plus-strand DNA at the central polypurine tract (cPPT) and central termination a the central termination sequence (CTS) lead to the formation of a three-stranded DNA structure: the HIV-I central DNA flap. While not wishing to be bound by any theory, the DNA flap may act as a cis-active determinant of lentiviral genome nuclear import and/or may increase the titer of the virus.

In one embodiment, retroviral or lentiviral transfer vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J Viral. 65: 1053; and Cullen et al., 1991. Cell 58: 423), and the hepatitis B virus post-transcriptional regulatory element (HPRE). Generally, the RNA export element is placed within the 3′ UTR of a gene, and may be inserted as one or multiple copies.

In other embodiments, expression of heterologous sequences in viral vectors is increased by incorporating post-transcriptional regulatory elements, efficient polyadenylation sites, and optionally, transcription termination signals into the vectors. A variety of posttranscriptional regulatory elements may increase expression of a heterologous nucleic acid at the protein, e.g., woodchuck hepatitis virus post-transcriptional regulatory element (WPRE; Zufferey et al., 1999, J Viral., 73 :2886); the post-transcriptional regulatory element present in hepatitis B virus (HPRE) (Huang et al., Mal. Cell. Biol., 5:3864); and the like (Liu et al., 1995, Genes Dev., 9:1766).

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “poly A site” or “poly A sequence” as used herein denotes a DNA sequence which directs both the termination and polyadenylation of the nascent RNA transcript by RNA polymerase II. Polyadenylation sequences may promote mRNA stability by addition of a poly A tail to the 3′ end of the coding sequence and thus, contribute to increased translational efficiency. Efficient polyadenylation of the recombinant transcript is desirable as transcripts lacking a poly A tail are unstable and are rapidly degraded. Illustrative examples of poly A signals that may be used in a vector of the disclosure, includes an ideal poly A sequence (e.g., AATAAA, ATTAAA, AGTAAA), a bovine growth hormone poly A sequence (BGHpA), a rabbit β-globin poly A sequence (rβgpA), or another suitable heterologous or endogenous poly A sequence known in the art.

The “control elements” or “regulatory sequences” present in an expression vector are those non-translated regions of the vector-origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters maybe used.

In particular embodiments, a vector for use in practicing the embodiments described herein including, but not limited to expression vectors and viral vectors, will include exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances may function independent of their orientation relative to another control sequence. An enhancer may function cooperatively or additively with promoters and/or other enhancer elements. The term “promoter/enhancer” refers to a segment of DNA which contains sequences capable of providing both promoter and enhancer functions.

The term “operably linked,” refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide—of interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments of the disclosure include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, HS, P7.5, and P11 promoters from vaccinia virus, an elongation factor I-alpha (EF1a) promoter, early growth response 1 (EGRI), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B 1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-I (PGK) promoter, a cytomegalovirus enhancer/chicken β-actin (CAG) promoter, a β-actin promoter and a myeloproliferative sarcoma virus enhancer, negative control region deleted, d1587rev primer-binding site substituted (MND) promoter (Challita et al., J Viral. 69(2):748-55 (1995)).

Additional examples of gene therapy that may be used in the present invention include, but are not limited to those described in U.S. Pat. No. 5,719,131 (cationic amphiphiles); U.S. Pat. No. 5,714,353 (retroviral vectors); U.S. Pat. No. 5,656,465 (non-integrating viruses, e.g., cytoplasmic viruses); U.S. Pat. Nos. 5,583,362; 5,399,346 (primary human cells, e.g., human blood cells used as vehicles for the transfer of human genes encoding therapeutic agents); U.S. Pat. No. 5,334,761 (cationic lipids useful for making lipid aggregates for delivery of macromolecules and other compounds into cells); U.S. Pat. No. 5,283,185 (cationic amphiphiles); U.S. Pat. No. 5,264,618 (cationic lipids); U.S. Pat. No. 5,252,479 (hybrid parvovirus vectors); U.S. Pat. No. 4,394,448 (DNA); each of which are incorporated herein by reference in their entirety.

Transfection of a cell with a gene therapy can be facilitated through the use of a carrier in combination with the gene therapy. Various different carriers have been developed for performing this function. Examples of different carriers which may be used include, but are not limited to, cationic lipids (derivatives of glycerolipids with a positively charged ammonium or sulfonium ion-containing headgroup, e.g., U.S. Pat. No. 5,711,964); cationic amphiphiles (e.g., U.S. Pat. Nos. 5,719,131; 5,650,096); cationic lipids (e.g., U.S. Pat. Nos. 5,527,928; 5,283,185; 5,264,618); and liposomes (e.g., U.S. Pat. Nos. 5,711,964; 5,705,385; 5,631,237), each of the U.S. Patents listed above being incorporated herein by reference.

Naked DNA is the simplest method of non-viral transfection and may be used in certain embodiments described herein.

In certain other embodiments, the use of oligonucleotides is also contemplated. The use of synthetic oligonucleotides in gene therapy is to inactivate the genes involved in the disease process. There are several methods by which this is achieved. One strategy uses antisense specific to the target gene to disrupt the transcription of the faulty gene. Another uses small molecules of RNA called siRNA to signal the cell to cleave specific unique sequences in the mRNA transcript of the faulty gene, disrupting translation of the faulty mRNA, and therefore expression of the gene. This is described in more detail below.

A further strategy uses double stranded oligodeoxynucleotides as a decoy for the transcription factors that are required to activate the transcription of the target gene. The transcription factors bind to the decoys instead of the promoter of the faulty gene, which reduces the transcription of the target gene, lowering expression.

To improve the delivery of the new DNA into the cell, the DNA must be protected from damage and its entry into the cell must be facilitated. To this end new molecules, lipoplexes and polyplexes, that have the ability to protect the DNA from undesirable degradation during the transfection process may be used in certain embodiments described herein.

In certain embodiments, plasmid DNA can be covered with lipids in an organized structure like a micelle or a liposome. When the organized structure is complexed with DNA it is called a lipoplex. There are three types of lipids, anionic (negatively charged), neutral, or cationic (positively charged).

Cationic lipids, due to their positive charge, naturally complex with the negatively charged DNA. Also as a result of their charge they interact with the cell membrane, endocytosis of the lipoplex occurs and the DNA is released into the cytoplasm. The cationic lipids also protect against degradation of the DNA by the cell.

Complexes of polymers with DNA are called polyplexes. Most polyplexes consist of cationic polymers and their production is regulated by ionic interactions. One large difference between the methods of action of polyplexes and lipoplexes is that polyplexes cannot release their DNA load into the cytoplasm, so to this end, co-transfection with endosome-lytic agents (to lyse the endosome that is made during endocytosis, the process by which the polyplex enters the cell) such as inactivated adenovirus must occur. However this is not always the case, polymers such as polyethylenimine have their own method of endosome disruption as does chitosan and trimethylchitosan.

Other methods relating to the use of viral vectors in gene therapy, which may be utilized according to certain embodiments of the present disclosure, may be found in, e.g., Kay, M. A. (1997) Chest 111(6 Supp.):138S-142S; Ferry, N. and Heard, J. M. (1998) Hum. Gene Ther. 9:1975-81; Shiratory, Y. et al. (1999) Liver 19:265-74; Oka, K. et al. (2000) Curr. Opin. Lipidol. 11:179-86; Thule, P. M. and Liu, J.M. (2000) Gene Ther. 7:1744-52; Yang, N. S. (1992) Crit. Rev. Biotechnol. 12:335-56; Alt, M. (1995) J Hepatol. 23:746-58; Brody, S. L. and Crystal, R. G. (1994) Ann. NY Acad. Sci. 716:90-101; Strayer, D. S. (1999) Expert Opin. Investig. Drugs 8:2159-2172; Smith-Arica, J. R. and Bartlett, J. S. (2001) Curr. Cardiol. Rep. 3:43-49; and Lee, H. C. et al. (2000) Nature 408:483-8.

In certain embodiments, the use of the RNA interference (RNAi) pathway that is used by cells to regulate the activity of many genes is contemplated. The term “RNA interference” (RNAi), also called post transcriptional gene silencing (PTGS), refers to the biological process in which RNA molecules inhibit gene expression.

In certain embodiments, an RNA interfering agent may be used in the described methods.

An “RNA interfering agent” as used herein, is defined as any agent that interferes with or inhibits expression of a target gene, e.g., a target gene of the invention, by RNA interference (RNAi). Such RNA interfering agents include, but are not limited to, nucleic acid molecules including RNA molecules, which are homologous to the target gene, e.g., a target gene of the invention, or a fragment thereof, short interfering RNA (siRNA), short hairpin RNA (shRNA), and small molecules which interfere with or inhibit expression of a target gene by RNA interference (RNAi).

“RNA interference (RNAi)” is a process whereby the expression or introduction of RNA of a sequence that is identical or highly similar to a target gene results in the sequence specific degradation or PTGS of messenger RNA (mRNA) transcribed from that targeted gene, thereby inhibiting expression of the target gene. RNAi can also be initiated by introducing nucleic acid molecules, e.g., synthetic siRNAs or RNA interfering agents, to inhibit or silence the expression of target genes. As used herein, “inhibition of target gene expression” or “inhibition of marker gene expression” includes any decrease in expression or protein activity or level of the target gene (e.g., a marker gene of the invention) or protein encoded by the target gene, e.g., a marker protein of the invention. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene. These are the effector molecules for inducing RNAi, leading to posttranscriptional gene silencing with RNA-induced silencing complex (RISC). In addition to siRNA, which can be chemically synthesized, various other systems in the form of potential effector molecules for posttranscriptional gene silencing are available, including short hairpin RNAs (shRNAs), long dsRNAs, short temporal RNAs, and micro RNAs (miRNAs). These effector molecules either are processed into siRNA, such as in the case of shRNA, or directly aid gene silencing, as in the case of miRNA. The present invention thus encompasses the use of shRNA as well as any other suitable form of RNA to effect posttranscriptional gene silencing by RNAi. Use of shRNA has the advantage over use of chemically synthesized siRNA in that the suppression of the target gene is typically long-term and stable. An siRNA may be chemically synthesized, may be produced by in vitro by transcription, or may be produced within a host cell from expressed shRNA.

In one embodiment, a siRNA is a small hairpin (also called stem loop) RNA (shRNA). These shRNAs are composed of a short (e.g., 19-25 nucleotides) antisense strand, followed by a 5-9 nucleotide loop, and the complementary sense strand. Alternatively, the sense strand may precede the nucleotide loop structure and the antisense strand may follow. These shRNAs may be contained in plasmids, retroviruses, and lentiviruses.

As used herein, “gene silencing” induced by RNA interference refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without introduction of RNA interference. In one preferred embodiment, the mRNA levels are decreased by at least about 70%, about 80%, about 90%, about 95%, about 99%, about 100%.

“Gene editing,” or “genome editing” with engineered nucleases is a type of genetic engineering in which DNA is inserted, deleted or replaced in the genome of an organism using engineered nucleases, or “molecular scissors.” These nucleases create site-specific double-strand breaks (DSBs) at desired locations in the genome. The induced double-strand breaks are repaired through nonhomologous end-joining (NHEJ) or homologous recombination (HR), resulting in targeted mutations (edits').

There are three families of engineered nucleases that may be used in certain embodiments described herein: Zinc finger nucleases (ZFNs), Transcription Activator-Like Effector-based Nucleases (TALENs), and CRISPR-Cas system.

“Zinc-finger nucleases” or “ZFNs” are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms. Alongside Cas9 and TALEN proteins, ZFN is becoming a prominent tool in the field of genome editing.

A zinc finger nuclease is a site-specific endonuclease designed to bind and cleave DNA at specific positions. There are two protein domains. The first domain is the DNA binding domain, which consists of eukaryotic transcription factors and contain the zinc finger. The second domain is the nuclease domain, which consists of the Fokl restriction enzyme and is responsible for the catalytic cleavage of DNA.

The DNA-binding domains of individual ZFNs typically contain between three and six individual zinc finger repeats and can each recognize between 9 and 18 basepairs. If the zinc finger domains are perfectly specific for their intended target site then even a pair of 3-finger ZFNs that recognize a total of 18 basepairs can, in theory, target a single locus in a mammalian genome. The most straightforward method to generate new zinc-finger arrays is to combine smaller zinc-finger “modules” of known specificity. The most common modular assembly process involves combining three separate zinc fingers that can each recognize a 3 basepair DNA sequence to generate a 3-finger array that can recognize a 9 basepair target site.

The non-specific cleavage domain from the type IIs restriction endonuclease Fokl is typically used as the cleavage domain in ZFNs. This cleavage domain must dimerize in order to cleave DNA and thus a pair of ZFNs are required to target non-palindromic DNA sites. Standard ZFNs fuse the cleavage domain to the C-terminus of each zinc finger domain. In order to allow the two cleavage domains to dimerize and cleave DNA, the two individual ZFNs must bind opposite strands of DNA with their C-termini a certain distance apart.

In certain embodiments, zinc finger nucleases may be useful to manipulate the genome of a subject, with the Fas receptor gene disrupted by zinc finger nucleases to be save as a potential treatment for many Fas mediated diseases, as described herein. Custom-designed ZFNs that combine the non-specific cleavage domain (N) of Fokl endonuclease with zinc-finger proteins (ZFPs) offer a general way to deliver a site-specific DSB to the genome, and stimulate local homologous recombination by several orders of magnitude. Since ZFN-encoding plasmids could be used to transiently express ZFNs to target a DSB to a specific gene locus in human cells, they offer an excellent way for targeted delivery of the therapeutic genes to a pre-selected chromosomal site.

In certain further embodiments, transcription activator-like effector nuclease (TALEN®) technology may be used in connection with the methods described herein, The TALEN® technology leverages artificial restriction enzymes generated by fusing a TAL effector DNA-binding domain to a DNA cleavage domain.

Restriction enzymes are enzymes that cut DNA strands at a specific sequence. Transcription activator-like effectors (TALEs) can be quickly engineered to bind practically any desired DNA sequence. By combining such an engineered TALE with a DNA cleavage domain (which cuts DNA strands), one can engineer restriction enzymes that will specifically cut any desired DNA sequence. When these restriction enzymes are introduced into cells, they can be used for gene editing or for genome editing in situ, a technique known as genome editing with engineered nucleases. Alongside zinc finger nucleases and Cas9 proteins, TALEN is becoming a prominent tool in the field of genome editing.

TAL effectors are proteins that are secreted by Xanthomonas bacteria. The DNA binding domain contains a repeated highly conserved 33-34 amino acid sequence with divergent 12th and 13th amino acids. These two positions, referred to as the Repeat Variable Diresidue (RVD), are highly variable and show a strong correlation with specific nucleotide recognition. This relationship between amino acid sequence and DNA recognition has allowed for the engineering of specific DNA-binding domains by selecting a combination of repeat segments containing the appropriate RVDs.

The non-specific DNA cleavage domain from the end of the Fokl endonuclease can be used to construct hybrid nucleases that are active in many different cell types. The Fokl domain functions as a dimer, requiring two constructs with unique DNA binding domains for sites in the target genome with proper orientation and spacing. Both the number of amino acid residues between the TALE DNA binding domain and the Fokl cleavage domain and the number of bases between the two individual TALEN binding sites appear to be important parameters for achieving high levels of activity.

The simple relationship between amino acid sequence and DNA recognition of the TALE binding domain allows for the efficient engineering of proteins. Once the TALEN constructs have been assembled, they are inserted into plasmids; the target cells are then transfected with the plasmids, and the gene products are expressed and enter the nucleus to access the genome. Alternatively, TALEN constructs can be delivered to the cells as mRNAs, which removes the possibility of genomic integration of the TALEN-expressing protein. Using an mRNA vector can also dramatically increase the level of homology directed repair (HDR) and the success of introgression during gene editing.

TALEN® technology can be used to edit genomes by inducing double-strand breaks (DSB), which cells respond to with repair mechanisms. Non-homologous end joining (NHEJ) reconnects DNA from either side of a double-strand break where there is very little or no sequence overlap for annealing. This repair mechanism induces errors in the genome via insertion or deletion, or chromosomal rearrangement; any such errors may render the gene products coded at that location non-functional. Because this activity can vary depending on the species, cell type, target gene, and nuclease used, it should be monitored when designing new systems. Alternatively, DNA can be introduced into a genome through NHEJ in the presence of exogenous double-stranded DNA fragments. Homology directed repair can also introduce foreign DNA at the DSB as the transfected double-stranded sequences are used as templates for the repair enzymes.

In certain embodiments, the TALEN® technology may be used to correct the genetic errors that underlie disease, such as inflammation-mediated and/or component mediated disease or condition. In theory, the genome-wide specificity of engineered TALEN fusions allows for correction of errors at individual genetic loci via homology-directed repair from a correct exogenous template.

In certain embodiments, the TALEN® technology may be combined with other genome engineering tools, such as meganucleases. The DNA binding region of a TAL effector can be combined with the cleavage domain of a meganuclease to create a hybrid architecture combining the ease of engineering and highly specific DNA binding activity of a TAL effector with the low site frequency and specificity of a meganuclease.

In certain further embodiments, Clustered regularly-interspaced short palindromic repeats (CRISPR) may be used in the methods of treatment of inflammation-mediated and/or component mediated diseases or conditions as described herein.

CRISPR are segments of prokaryotic DNA containing short repetitions of base sequences. CRISPR may be used to edit genomes with unprecedented precision, efficiency, and flexibility.

The CRISPR/Cas system is a prokaryotic immune system that confers resistance to foreign genetic elements such as plasmids and phages, and provides a form of acquired immunity. CRISPR spacers recognize and cut these exogenous genetic elements in a manner analogous to RNA interference in eukaryotic organisms. A set of genes was found to be associated with CRISPR repeats, and was named the cas, or CRISPR-associated, genes. The cas genes encode putative nuclease or helicase proteins, which are enzymes that can cut or unwind DNA. The Cas genes are always located near the CRISPR sequences. There are a number Cas enzymes, but the best known is called Cas9, which comes from Streptococcus pyogenes. By delivering the Cas9 protein and appropriate guide RNAs into a cell, the organism's genome can be cut at any desired location.

Like RNAi, CRISPR interference (CRISPRi) turns off genes in a reversible fashion by targeting, but not cutting a site. The targeted site is methylated so the gene is epigenetically modified. This modification inhibits transcription. Cas9 is an effective way of targeting and silencing specific genes at the DNA level. For instance, CRISPR may be applied to cells to introduce targeted mutations in genes relevant to a specific disease or condition.

Transfection of a cell with a gene therapy agent can be facilitated through the use of a carrier in combination with the gene therapy agent. Various different carriers have been developed for performing this function. Examples of different carriers which may be used include, but are not limited to, cationic lipids (derivatives of glycerolipids with a positively charged ammonium or sulfonium ion-containing headgroup; e.g., U.S. Pat. No. 5,711,964); cationic amphiphiles (e.g., U.S. Pat. Nos. 5,719,131; 5,650,096); cationic lipids (e.g., U.S. Pat. Nos. 5,527,928; 5,283,185; 5,264,618); and liposomes (e.g., U.S. Pat. Nos. 5,711,964; 5,705,385; 5,631,237), each of the U.S. Patents listed above being incorporated herein by reference.

### Compositions

Certain embodiments relate to compositions that include the described Fas inhibitor(s), a derivative, fragment, a pharmaceutically acceptable salt thereof, or a gene therapy encoding the described Fas inhibitor in an amount effective to inhibit Fas signaling.

The composition may be a “pharmaceutical composition,” a “pharmaceutical preparation,” or a “pharmaceutical formulation.”

As used herein, the term “pharmaceutical composition” refers to the combination of one or more pharmaceutical agents (e.g., Fas inhibitor) with one or more carriers, inert or active, making the composition especially suitable for diagnostic or therapeutic use in vitro, in vivo or ex vivo. A pharmaceutical composition comprises the physical entity that is administered to a subject, and may take the form of a solid, semi-solid or liquid dosage form, such as tablet, capsule, orally-disintegrating tablet, pill, powder, suppository, solution, elixir, syrup, suspension, cream, lozenge, paste, spray, etc. A pharmaceutical composition may comprise a single pharmaceutical formulation (e.g., extended release, immediate release, delayed release, nanoparticulate, etc.) or multiple formulations (e.g., immediate release and delayed release, nanoparticulate and non-nanoparticulate, etc.).

As used herein, the terms “pharmaceutical preparation” or “pharmaceutical formulation” refer to at least one, but may be two, three or more, pharmaceutical agent(s) (e.g., Fas inhibitor, e.g., Met, Met-12 or Compound 1) in combination with one or more additional components that assist in rendering the pharmaceutical agent(s) suitable for achieving the desired effect upon administration to a subject. The pharmaceutical formulation may include one or more additives, for example pharmaceutically acceptable excipients, carriers, penetration enhancers, coatings, stabilizers, buffers, acids, bases, or other materials physically associated with the pharmaceutical agent to enhance the administration, release (e.g., timing of release), deliverability, bioavailability, effectiveness, etc. of the dosage form. The formulation may be, for example, a liquid, a suspension, a solid, a nanoparticle, emulsion, micelle, ointment, gel, emulsion, coating, etc. A pharmaceutical formulation may contain a single pharmaceutical agent (e.g., Met, Met-12 or Compound 1) or multiple pharmaceutical agents. A pharmaceutical composition may contain a single pharmaceutical formulation or multiple pharmaceutical formulations. In some embodiments, a pharmaceutical agent (e.g., Met, Met-12 or Compound 1) is formulated for a particular mode of administration (e.g., ocular administration (e.g., intravitreal administration, etc.), etc.). A pharmaceutical formulation is sterile, non-pyrogenic and non-toxic to the subject. The terms “pharmaceutical composition” and “pharmaceutical formulation” may be used interchangeably.

Certain embodiments, relate to compositions that include the described Fas inhibitor, a derivative, or a pharmaceutically acceptable salt thereof; and a pharmaceutically acceptable additive. The additive may be selected from carriers, excipients, disintegrators or disintegrating aids, binders, lubricants, coating agents, pigments, diluents, bases, dissolving agents or solubilizers, isotonic agents, pH regulators, stabilizers, propellants, adhesives, and other additives known in the art.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. Pharmaceutically acceptable carriers include carbohydrates such as trehalose, mannitol, xylitol, sucrose, lactose, and sorbitol. Other ingredients for use in formulations may include DPPC, DOPE, DSPC and DOPC. Natural or synthetic surfactants may be used. PEG may be used (even apart from its use in derivatizing the protein or analog). Dextrans, such as cyclodextran, may be used. Bile salts and other related enhancers may be used. Cellulose and cellulose derivatives may be used. Amino acids may be used, such as use in a buffer formulation.

For further examples of carriers, stabilizers and adjuvants see, e.g., Martin, Remington's Pharmaceutical Sciences, 15th Ed., Mack Publ. Co., Easton, Pa. [1975]; herein incorporated by reference in its entirety.

Also, the use of liposomes, microcapsules or microspheres, inclusion complexes, or other types of carriers known in the art, is contemplated.

In certain embodiments, the composition may include at least one non-ionic surfactant. Examples of non-ionic surfactants include Polysorbate 80, Polysorbate 20, Poloxamer 407, and Tyloxapol.

The composition may be in any form suitable for administration to a subject, e.g., solution, pill, ointment, suspension, eye drops, gel, cream, foam, spray, liniment, and powder. As used herein, the term “administration” refers to the act of giving a drug, prodrug, or other agent, or therapeutic treatment (e.g., Fas inhibitor and/or compositions thereof described herein) to a subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, rectal, by injection (e.g., intravenously, subcutaneously, intratumorally, intraperitoneally, intravitreally, periocularlly, etc.) and the like. Implantable sustained release forms/formulations are also contemplated.

The compositions and methods described herein are particularly applicable for human subjects at risk for or suffering from inflammation-mediated and/or complement-mediated disease or condition, such as retinal disease (e.g., glaucoma, retinal detachment, AMD (dry and wet), diabetic retinopathy, Uveitis, retinal vein occlusion, retinitis pigmentosa or NAION), immunological disease, cancer, amyloid disease (e.g., Alzheimer's disease, type-2 diabetes, Huntington's disease, ALS, or Parkinson's disease), autoimmune disease (e.g., allergy, lupus, or rheumatoid arthritis), an injury caused by ischemia or reperfusion (e.g., stroke), neurodegeneration, and diseases of the central nervous system. The etiology of the disease or condition, itself, may or may not be Fas-mediated, but Fas-mediated signaling through one or more signaling pathways accelerates or amplifies disease symptoms and/or severity.

The compositions for topical use could be in any form deemed suitable by the person skilled in the art to be applied directly on the ocular surface, like e.g., solution, ointment, suspension, eye drops, gel, cream, foam, spray, liniment, powder.

The Fas inhibitor or a composition thereof may administered daily (once, twice, 3 times, 4 times/day, etc.), every other day, every 3 days, weekly, biweekly, monthly, bimonthly, or tri-monthly, etc.

The described Fas inhibitors or compositions thereof may be administered in an amount effective to inhibit Fas and/or Fas signaling. The term “an amount effective” means an amount of a drug or agent (e.g., Compound 1) or its' formulation effective to facilitate a desired therapeutic effect (e.g., inhibition of Fas signaling) in a particular class of subjects (e.g., infant, child, adolescent, adult). U.S. Food and Drug Administration (FDA) recommended dosages are indicative of a therapeutic dose. For example, in the context of this application, the desired therapeutic effect may be preventing or treating inflammation-mediated and/or complement-mediated disease or condition or limiting the severity of inflammation-mediated and/or complement-mediated disease or condition.

For example, an effective amount may be a daily dose of Fas inhibitor in a range, e.g., from about 1 ng to about 1 mg.

In one embodiment, the composition is in the form of eye drops and the described Fas inhibitor is in a concentration between 0.000001% w/v and 2% w/v.

In certain embodiments, compositions comprise one or more additives, such as carriers, diluents and/or excipients suitable for preparing, e.g., ophthalmic compositions. Suitable for preparing ophthalmic compositions are all carriers, diluents or excipients tolerated by the eye. Examples of excipients that may be used in said compositions are Polysorbate 80, polyethylene glycol (e.g., PEG200, PEG400) dextran and the like.

The compositions may comprise carriers for improving the Fas inhibitor's bioavailability by increasing corneal permeability, like e.g. dimethyl sulfoxide, membrane phospholipids and surfactants.

In certain embodiment, such compositions may also comprise carriers apt to increase bioavailability, stability and tolerability of the active principle. For instance, viscosity-increasing agents such as hyaluronic acid, methylcellulose, polyvinyl alcohol, polyvinyl pyrrolidone, etc. may be used.

To prevent contaminations, the described compositions could comprise one or more preservatives having antimicrobial activity, like e.g. benzalchonium chloride (shortened in BAK).

### Uses and Methods

In certain embodiments, the described Fas inhibitors may be used for preventing, treating or ameliorating an inflammation-mediated and/or complement-mediated disease or condition in a subject.

Examples of diseases or conditions that may be treated with the described Fas inhibitors include, e.g., retinal disease (e.g., glaucoma, retinal detachment, AMD (dry and wet), diabetic retinopathy, Uveitis, retinal vein occlusion, inherited retinal degeneration diseases including retinitis pigmentosa, or NAION), immunological disease, cancer, amyloid disease (e.g., Alzheimer's disease, type-2 diabetes, Huntington's disease, ALS, or Parkinson's disease), traumatic injury (e.g. traumatic brain injury), autoimmune disease (e.g., allergy, lupus, or rheumatoid arthritis), an injury caused by ischemia or reperfusion (e.g., stroke), neurodegeneration, and diseases of the central nervous system (e.g., neuropathies and demyelinating diseases such as multiple sclerosis and inflammatory demyelinating diseases).

Certain embodiments relate to methods of inhibiting Fas signaling to prevent, treat, or ameliorate inflammation-mediated and/or complement-mediated diseases or conditions.

Surprisingly, without being bound by the mechanism of action, it was discovered that the inhibition of Fas/Fas signaling results in at least one of the following: reduction of expression or concentration of at least one Fas-mediated inflammation-related gene or protein; reduction of expression or concentration of at least one Fas-mediated complement-related gene or protein, including complement component 3 (C3) and complement component 1q (C1q); reduction of gene or protein expression or concentration of Caspase 8; reduction of gene or protein expression or concentration of one or more components of the inflammasome, including NLRP3 and NLRP2; reduction of gene or protein expression or concentration of one or more C—X—C motif chemokines, including CXCL2 (MIP-2α) and CXCL10 (IP-10); reduction of gene or protein expression or concentration of one or more C—X3-C motif chemokines, including CX3CL1 (fractalkine); reduction of gene or protein expression or concentration of one or more C—C motif chemokines, including CCL2 (MCP-1), CCL3 (MIP-1α), and CCL4 (MIP-1β); reduction of gene or protein expression or concentration of toll-like receptor 4 (TLR4); reduction of gene or protein expression or concentration of one or more interleukin cytokines, including IL-1β, IL-18, and IL-6; reduction of gene or protein expression or concentration of one or more TNF superfamily cytokines, including TNFα; reduction of Fas-mediated Muller cell activation as indicated by reduced GFAP gene or protein expression or concentration; or increase of expression or concentration or prevent the reduction of expression or concentration of at least one pro-survival gene or protein (e.g., cFLIP). The term “Fas-mediated” means involving or depending on the Fas receptor and/or its activation.

As such, certain embodiments relate to a method for preventing, treating, or ameliorating inflammation-mediated and/or complement-mediated disease or condition in a subject including administering to the subject the described Fas inhibitor or a derivative thereof, or a fragment thereof, or a gene therapy encoding the Fas inhibitor in an amount effective to inhibit Fas and/or Fas signaling, and thereby ameliorate or prevent the disease or condition in the subject, wherein the inhibition of Fas and/or Fas signaling results in at least one (or at least two, or at least three, etc., or all) of the following: reduction of expression or concentration of at least one Fas-mediated inflammation-related gene or protein (e.g.,TNFα, IL-1β, IP-10, IL-18, MIP1α, IL-6, GFAP, MIP2, MCP-1, or MIP-1β); reduction of expression or concentration of at least one Fas-mediated complement-related gene or protein (e.g., complement component 3 (C3) and complement component 1q (C1q)); reduction of gene or protein expression or concentration of Caspase 8; reduction of gene or protein expression or concentration of one or more components of the inflammasome (e.g., NLRP3 and NLRP2); reduction of gene or protein expression or concentration of one or more C—X—C motif chemokines (e.g., CXCL2 (MIP-2α) and CXCL10 (IP-10)); reduction of gene or protein expression or concentration of one or more C—X3-C motif chemokines (e.g., CX3CL1 (fractalkine)); reduction of gene or protein expression or concentration of one or more C-C motif chemokines (e.g., CCL2 (MCP-1), CCL3 (MIP-1α), and CCL4 (MIP-1β)); reduction of gene or protein expression or concentration of toll-like receptor 4 (TLR4); reduction of gene or protein expression or concentration of one or more interleukin cytokines (e.g., IL-1β, IL-18, and IL-6); reduction of gene or protein expression or concentration of one or more TNF superfamily cytokines (e.g., TNFα); reduction of Fas-mediated Muller cell activation as indicated by reduced GFAP gene or protein expression or concentration; or increase of expression or concentration or prevent the reduction of expression or concentration of at least one pro-survival gene or protein (e.g., cFLIP). The Fas inhibitor may be selected from the group consisting of: Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or gene therapy agents encoding the Fas inhibitor. The subject may have or is at risk of having the inflammation-mediated and/or complement-mediated disease or condition

The inflammation-mediated and/or complement-mediated disease or condition may be a retinal disease, immunological disease, cancer, amyloid disease, an injury caused by ischemia or reperfusion, an injury caused by trauma, neurodegeneration, and diseases of the central nervous system. Examples of the amyloid disease include Alzheimer's disease, type-2 diabetes, Huntington's disease, ALS, or Parkinson's disease. An example of the injury by ischemia or reperfusion is stroke. An example of the injury by trauma is traumatic brain injury.

Exemplary autoimmune diseases include allergies, lupus, and rheumatoid arthritis. Exemplary retinal diseases include glaucoma, retinal detachment, AMD (dry and wet), diabetic retinopathy, Uveitis, retinal vein occlusion, inherited retinal degeneration including retinitis pigmentosa, and NAION. Examples of diseases of the central nervous system include neuropathy or a demyelinating disease selected from the group consisting of multiple sclerosis and inflammatory demyelinating diseases.

In the described methods, the Fas inhibitor, its derivative, fragment, the gene therapy product, its corresponding interfering RNA (RNAi), or the pharmaceutically acceptable salt thereof may be administered in a pharmaceutical composition comprising the Fas inhibitor, its derivative, fragment, pharmaceutically acceptable salt, or a gene therapy that encodes the Fas inhibitor; and a pharmaceutically acceptable additive, such as carriers, excipients, disintegrators or disintegrating aids, binders, lubricants, coating agents, pigments, diluents, bases, dissolving agents or solubilizers, isotonic agents, pH regulators, stabilizers, propellants, and adhesives.

In the described methods, the Fas inhibitor, its derivative, or the pharmaceutically acceptable salt thereof may be administered via an injection.

A further embodiment relates to a method for preventing, treating or ameliorating an inflammation-mediated and/or complement-mediated disease or condition in a subject comprising administering to the subject a Fas inhibitor selected from the group consisting of Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor, in an amount effective to inhibit Fas signaling, and thereby prevent, treat or ameliorate the inflammation-mediated and/or complement-mediated disease or condition in the subject. The subject has or is at risk of having the inflammation-mediated and/or complement-mediated disease or condition. The inflammation-mediated and/or complement-mediated disease or condition may be retinal disease (e.g., glaucoma, retinal detachment, AMD (dry and wet), diabetic retinopathy, Uveitis, retinal vein occlusion, inherited retinal degenerations, including retinitis pigmentosa, or NAION), immunological disease, cancer, amyloid disease (e.g., Alzheimer's disease, type-2 diabetes, Huntington's disease, ALS, or Parkinson's disease), an injury caused by ischemia or reperfusion (e.g., stroke), autoimmune disease (e.g., allergy, lupus, or rheumatoid arthritis), neurodegeneration, and diseases of the central nervous system (e.g., neuropathy or a demyelinating disease selected from the group consisting of multiple sclerosis and inflammatory demyelinating diseases). The Fas inhibitor may be administered in a pharmaceutical composition comprising the Fas inhibitor and a pharmaceutically acceptable additive selected from the group consisting of carriers, excipients, disintegrators or disintegrating aids, binders, lubricants, coating agents, pigments, diluents, bases, dissolving agents or solubilizers, isotonic agents, pH regulators, stabilizers, propellants, and adhesives. The Fas inhibitor may be administered via an injection (e.g., an intravitreal injection, intrathecal, intravenous, or periocular injection).

Another embodiment related to a method for preserving retinal ganglion cells and axon density, or preventing the loss of ganglion cells and axon density in a patient with glaucoma comprising administering to the subject a Fas inhibitor, a derivative thereof, a fragment thereof, a pharmaceutically acceptable salt thereof, or a gene therapy encoding the Fas inhibitor, wherein the preserving or preventing the loss of retinal ganglion cells and axon density, or preventing the loss thereof is due to at least one (or at least two, or all three) of the following: inhibition of microglial/macrophage activation or recruitment; inhibition of at least one of TNF-α, CCL2/MCP-1 or CCL3/MIP-1α gene or protein expression or concentration; or reduction of IL-1β gene or protein expression or protein maturation, wherein the Fas inhibitor is administered to the subject in an amount effective to inhibit Fas signaling. The Fas inhibitor, a derivative thereof, a fragment thereof, a pharmaceutically acceptable salt thereof, or a gene therapy encoding the Fas inhibitor may be administered in a pharmaceutical composition comprising the Fas inhibitor, a derivative thereof, a fragment thereof, a pharmaceutically acceptable salt thereof, or a gene therapy encoding the Fas inhibitor; and a pharmaceutically acceptable additive. The additive may be selected from the group consisting of carriers, excipients, disintegrators or disintegrating aids, binders, lubricants, coating agents, pigments, diluents, bases, dissolving agents or solubilizers, isotonic agents, pH regulators, stabilizers, propellants and adhesives. The composition may be in a form selected from the group consisting of: solution, pill, ointment, suspension, eye drops, gel, cream, foam, spray, liniment, and powder. The administering may be via an injection, wherein the injection is an intravitreal injection, intrathecal, intravenous or periocular injection. The composition may further comprise at least one non-ionic surfactant selected from the group consisting of Polysorbate 80, Polysorbate 20, Poloxamer 407, and Tyloxapol. The Fas inhibitor or the composition comprising the Fas inhibitor may be administered daily, twice daily, every other day, weekly, biweekly, monthly, bimonthly, or tri-monthly. The Fas inhibitor or the composition comprising Fas inhibitor may be administered in a daily dose of from about 1 ng to about 1 mg. The composition may be in the form of eye drops and the Fas inhibitor is in a concentration between 0.000001% w/v and 2% w/v.

Yet another embodiment relates to a method of treating a subject having an increase (e.g., at least 5%, or at least 10%, etc.) in the mRNA and/or protein expression level(s) of at least one (or at least two, or at least three, etc., or all) of the following gene and/or protein in the subject's eye, as compared to a control: at least one Fas-mediated inflammation-related gene or protein (e.g. TNFα, IL-1β, IP-10, IL-18, MIP1α, IL-6, GFAP, MIP2, MCP-1, or MIP-1β); at least one Fas-mediated complement-related gene or protein (complement component 3 (C3) or complement component 1q (C1q)); Caspase 8; one or more components of the inflammasome (e.g., NLRP3 or NLRP2); one or more C—X—C motif chemokines (e.g., CXCL2 (MIP-2α) or CXCL10 (IP-10)); one or more C—X3-C motif chemokines (e.g., CX3CL1 (fractalkine)); one or more C—C motif chemokines (CCL2 (MCP-1), CCL3 (MIP-1α), and CCL4 (MIP-1β)); toll-like receptor 4 (TLR4); one or more interleukin cytokines (e.g., IL-1β, IL-18, and IL-6); one or more TNF superfamily cytokines (e.g., TNFα); or GFAP gene or protein expression or concentration, the method comprising administering to the subject a Fas inhibitor. The Fas inhibitor may be any Fas inhibitor described herein. For example, the Fas inhibitor may be selected from the group consisting of: Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor.

Yet further embodiment relates to a method of treating a subject having an increase (e.g., at least a 5%, or at least 10%, etc.) in the mRNA and/or protein expression level(s) of at least one (or at least two, or at least three, etc., or all) of the following gene and/or protein in the subject's serum, plasma, whole blood, or cerebrospinal fluid, as compared to a control: at least one Fas-mediated inflammation-related gene or protein (e.g. TNFα, IL-1β, IP-10, IL-18, MIP1α, IL-6, GFAP, MIP2, MCP-1, or MIP-1β); at least one Fas-mediated complement-related gene or protein (complement component 3 (C3) or complement component 1q (C1q)); Caspase 8; one or more components of the inflammasome (e.g., NLRP3 or NLRP2); one or more C—X—C motif chemokines (e.g., CXCL2 (MIP-2α) or CXCL10 (IP-10)); one or more C—X3-C motif chemokines (e.g., CX3CL1 (fractalkine)); one or more C-C motif chemokines (CCL2 (MCP-1), CCL3 (MIP-1α), and CCL4 (MIP-1β)); toll-like receptor 4 (TLR4); one or more interleukin cytokines (e.g., IL-1β, IL-18, and IL-6); one or more TNF superfamily cytokines (e.g., TNFα); or GFAP gene or protein expression or concentration, the method comprising administering to the subject a Fas inhibitor, the method comprising administering to the subject a Fas inhibitor. The Fas inhibitor may be any Fas inhibitor described herein. For example, the Fas inhibitor may be selected from the group consisting of: Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor.

In certain embodiments, the described compositions may include a pharmaceutical drug or agent. As used herein, the terms “pharmaceutical drug” or “pharmaceutical agent” refer to a compound, peptide, macromolecule, gene therapy agents, nucleic acids, or other entity that is administered (e.g., within the context of a pharmaceutical composition) to a subject to elicit a desired biological response. A pharmaceutical agent may be a “drug” or any other material (e.g., peptide, polypeptide, nucleic acid), which is biologically active in a human being or other mammal, locally and/or systemically. Examples of drugs are disclosed in the Merck Index and the Physicians Desk Reference, the entire disclosures of which are incorporated by reference herein for all purposes.

Treatment in vivo, i.e., by a method where Fas inhibitor (e.g., Met, Met-12 or Compound 1) is administered to a patient, is expected to result in preventing, treating, or ameliorating an inflammation-mediated and/or complement-mediated disease or condition.

It was surprisingly discovered that expression of inflammation-related genes was significantly reduced in animals treated with Compound 1 as compared to the controls. Also, the gene expression of the complement-related proteins was significantly reduced following the treatment with Compound 1. Even more surprisingly, the expression of cFLIP, generally considered to be pro-survival, was decreased in the control animals, and restored to near-baseline in the Compound 1 treated animals.

These data demonstrate that Fas inhibition by Compound 1 reduces the expression of inflammatory genes following elevated IOP, thereby preventing and/or reducing the inflammatory microenvironment induced by elevated IOP. Additionally, the observation that the expression of complement factors C3 and C1q were significantly elevated with microbead injection and were significantly reduced with Compound 1 treatment, suggests that Fas is upstream of complement signaling.

Taken together, these observations suggest that Fas is upstream of a host of inflammatory mediators, and inhibition of one of these downstream factors may not prevent the overall inflammatory microenvironment as effectively as inhibiting Fas.

In view of this, certain embodiments relate to a method for inhibiting Fas as part of a therapeutic strategy for treatment of inflammation-mediated and/or complement-mediated conditions and/or disorders, including glaucoma.

### EXAMPLES

### Example 1

The goal of this study was to analyze the tissue samples for changes in gene expression following elevation of intraocular pressure (“IOP”) in the presence or absence of Compound 1.

Methods:

Quantitative PCR (qPCR) was used on neural retina samples isolated at 28 days post microbead or saline injection from mice treated with Compound 1 (or vehicle) on Day 0.

Data shown are mRNA expression fold change over saline+vehicle control+/−SEM. N=6/group, **P<0.01, ***P<0.001, ****P<0.0001.

A 96-well was expanded to a 384-well qPCR system to allow for an increase in the number of genes to be examined in one run.

Also, new house keeping genes were tested and validated, as the house keeping genes, beta actin and HPRT1, that were used in our previous studies, proved to be unreliable and showed variable expression levels between our experimental groups.

After testing several retina house keeping genes, it was found that B2-microglobulin (B2M) and peptidylprolyl isomerase A (PPIA) were both very stable between all experimental groups and the average Ct-values for both house keeping genes were used to calculate DCt in these studies.

For this qPCR analysis, saline +vehicle was used as the control. DDCt=experimental DCt−mean DCt of saline+vehicle, and Expression Fold Change=2\hat{ }-DDCt.

Results:

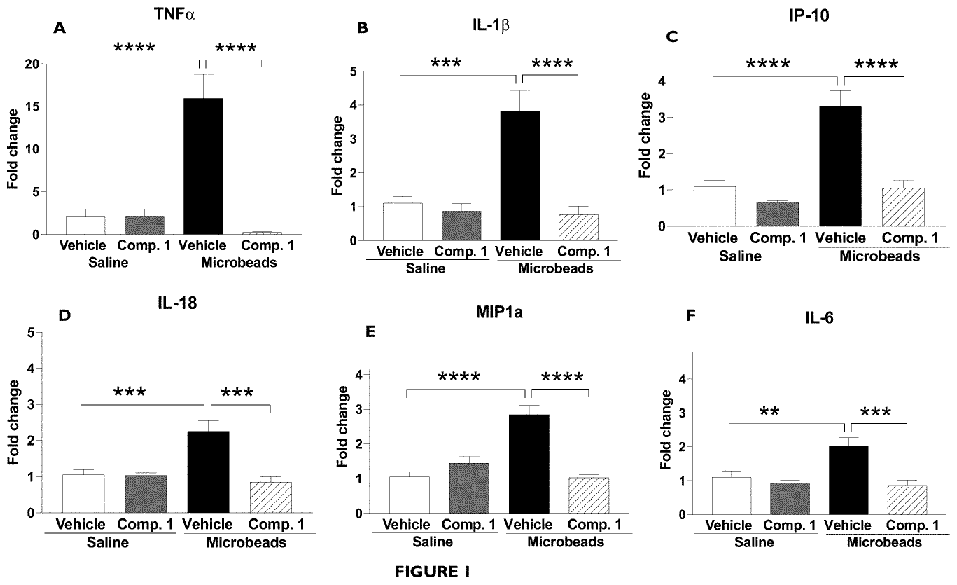

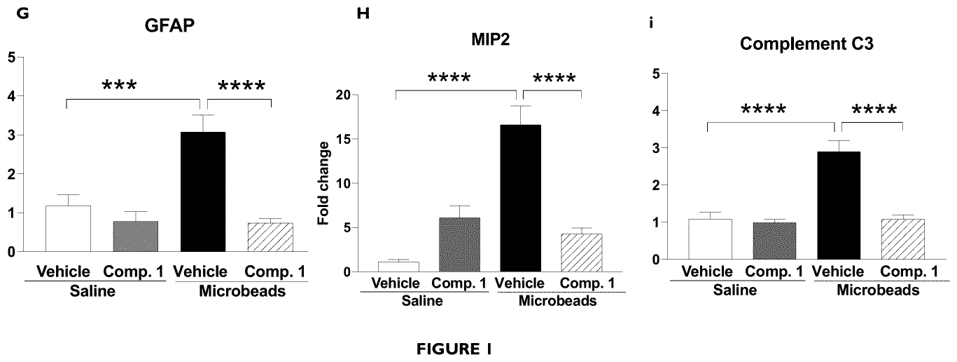

As shown in FIGS. 1 and 2, animals that were injected with microbeads and vehicle exhibited significantly higher expression of the inflammation-related genes, TNFα (FIG. 1A), IL-1β (FIG. 1B), IP-10 (FIG. 1C), IL-18 (FIG. 1D), MIP1α (FIG. 1E), IL-6 (FIG. 1F), GFAP (FIG. 1G), MIP2 (FIG. 1H), MCP-1 (FIG. 2A), and MIP-1β (FIG. 2E). The expression of these genes was significantly reduced in animals treated with Compound 1.

Following elevated IOP, the gene expression of the complement-related proteins C1q (FIG. 2G) and Complement C3 (FIG. 1i) were also significantly increased. As seen with the expression of the other inflammatory genes, the expression of C3 and C1q was significantly reduced in the animals treated with Compound 1.

Additional genes (Caspase 8, NLRP3, TLR4) were increased following elevated IOP (see FIG. 2B, FIG. 2F and FIG. 2D), but the increase did not reach significance when comparing the microbead group to the saline control group. However, the expression of all three genes were significantly reduced in the microbead injected animals treated with Compound 1.

The expression of cFLIP (FIG. 2C), generally considered to be pro-survival, was decreased in the microbead/saline animals, and restored to near-baseline in the Compound 1 treated animals.

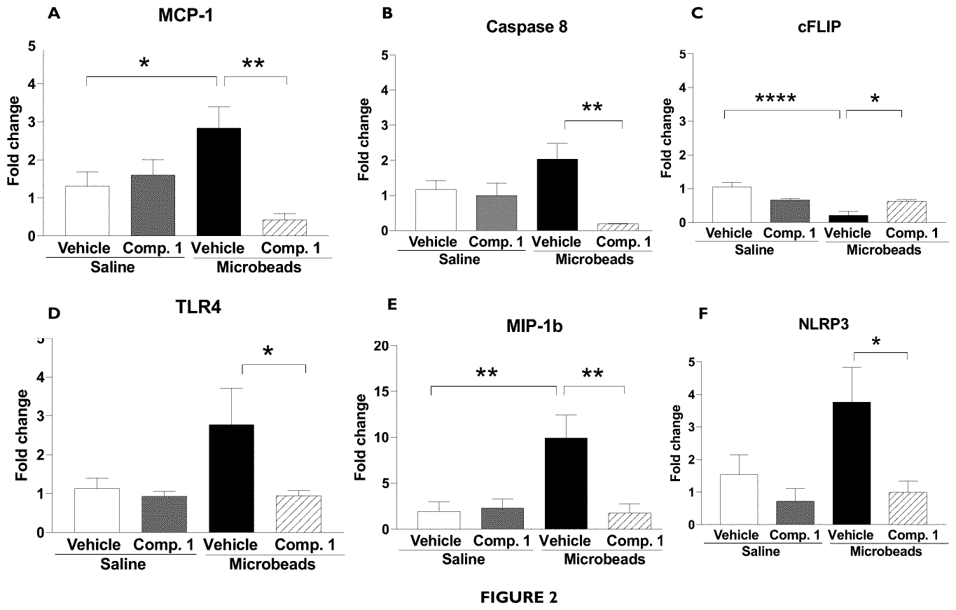

As shown in FIG. 3, other genes related to apoptosis, including Bax (FIG. 3A), FADD (FIG. 3B), FasR (FIG. 3D), FasL (FIG. 3E), and caspase 3 (FIG. 3H), were unchanged following elevated TOP and appeared to be unaffected by Compound 1 at this 28 day time point. Limited or no change was observed in some other inflammation-related genes, including ASC (FIG. 3C), NLRP2 (FIG. 3G), and complement C4 (FIG. 3F).

As described previously, Fas has been known to induce inflammatory signaling that propagate cell death and tissue damage. These data demonstrate that Fas inhibition by Compound 1 reduces the expression of inflammatory genes following elevated IOP, thereby preventing and/or reducing the inflammatory microenvironment induced by elevated IOP.

Additionally, the observation that the expression of complement factors C3 and C1q were significantly elevated with microbead injection and were significantly reduced with Compound 1 treatment, suggests that Fas is upstream of complement signaling.

Taken together, these observations suggest that Fas is upstream of a host of inflammatory mediators, and inhibition of one of these downstream factors may not prevent the overall inflammatory microenvironment as effectively as inhibiting Fas.

These data support the potential of Compound 1 and Fas inhibition as part of a therapeutic strategy for treatment of glaucoma.

### Example 2

The goals of this study were to determine whether the Fas inhibitor, Compound 1 can prevent the death of retinal ganglion cells (RGCs) and axons in the microbead-induced mouse model of elevated IOP and to evaluate if Fas inhibition can down-modulate the inflammatory microenvironment.

Methods:

All animal experiments were approved by the Institutional Animal Care and Use Committee at Schepens Eye Research Institute and were performed under the guidelines of the Association of Research in Vision and Ophthalmology (Rockville, Md.).

C57BL/6J mice were used in this experiment in which 2 μL of sterile polystyrene microbeads (15 μm; 7.2×10⁶ bead/mL) or saline were injected into the anterior chamber on Day 0 followed by 1 μL of 0.5 mg/mL or 2 mg/mL Compound 1 or vehicle by intravitreal (IVT) injection on Day 0 or 7 days after the microbead/saline injections. IOP was followed every 3 days for 4 weeks using a rebound tonometer (TonoLab). At 4 weeks post anterior chamber injection, retinal flatmounts were prepared and stained for Brn3a, an RGC-specific protein, to visualize RGCs. Sixteen non-overlapping images were taken, at 60× with 4-5 images within each quadrant and the images were used to calculate the RGC density. For axon analysis, optic nerves were stained with p-phenylenediamine (PPD) to visualize myelinated axons and 10 non-overlapping photomicrographs were taken at 100× magnification covering the entire area of the optic nerve cross-section, and these images were used to calculate the axon density. Quantitative PCR (qPCR) was also performed on retinal tissue isolated from the mice at 28 days post microbead/saline injections. To assess production of mature IL-1β (p17), protein lysates (20 μg per sample) were prepared from posterior eye cups (neural retina, choroid, and sclera) at 28 days post microbead/saline injections and analyzed by Western blot and densitometry. All data are presented as mean±SEM. One-way ANOVA and the Sidak multiple-comparison test were used for analysis of RGCs and axons. A p value <0.05 was considered significant.

Results:

IOP:

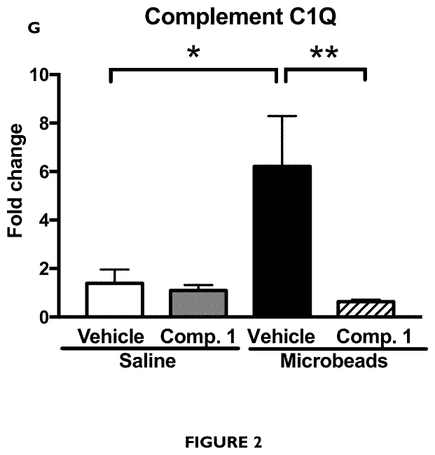

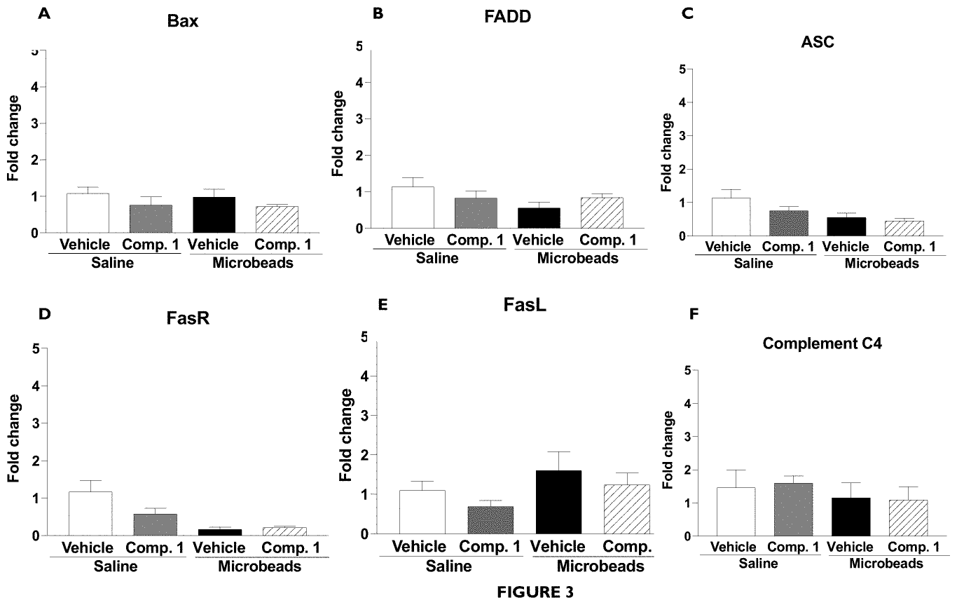

The microbead injections induced the expected increase in IOP to 20-25 mm Hg from a baseline of 15mm Hg, peaking around day 3 or 7 post-microbead injection. Saline injection had no significant effect on IOP. IVT injection with Compound 1 did not affect IOP when administered on the same day as the microbeads (FIG. 4) or when administered on Day 7 post microbeads (FIG. 5).

RGC and Axon Counts:

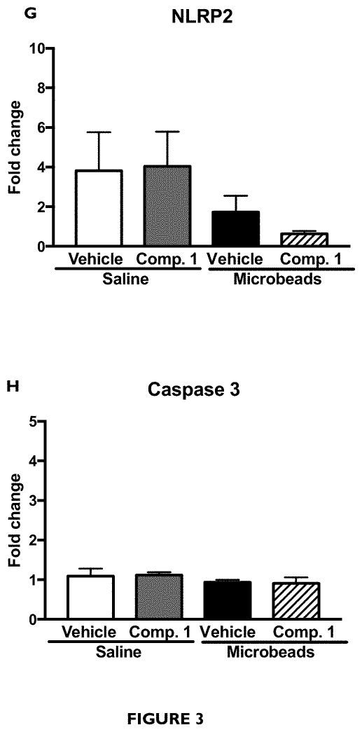

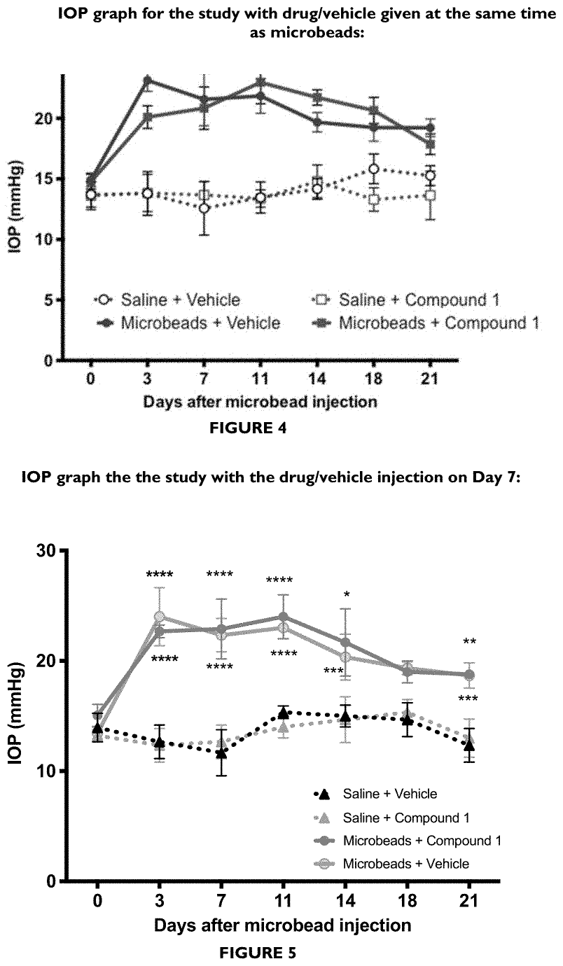

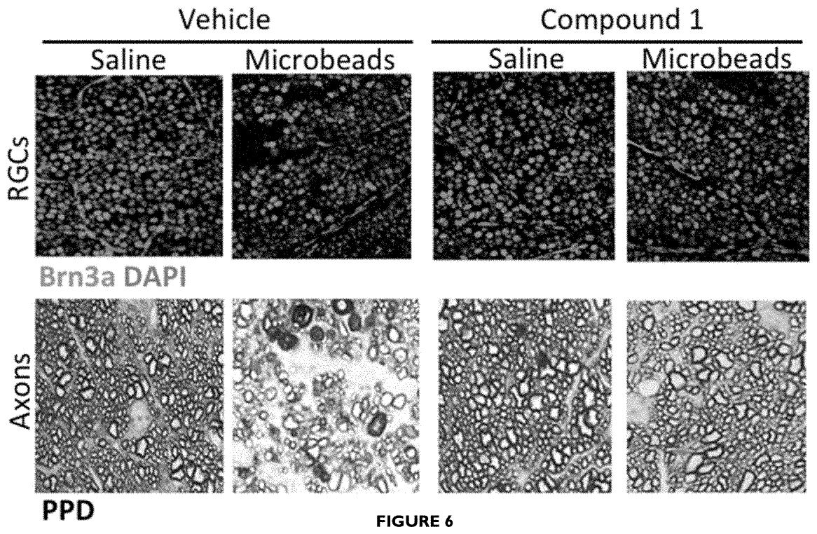

Treatment with Compound 1 at 0.5 mg/ml or 2 mg/ml achieved comparable and statistically significant preservation of retinal ganglion cell and axon density when given at Day 0. Representative images (FIG. 6) and the quantification of the total collected images are shown in FIG. 7 for RGC Cell density and FIG. 8 for Axon density.

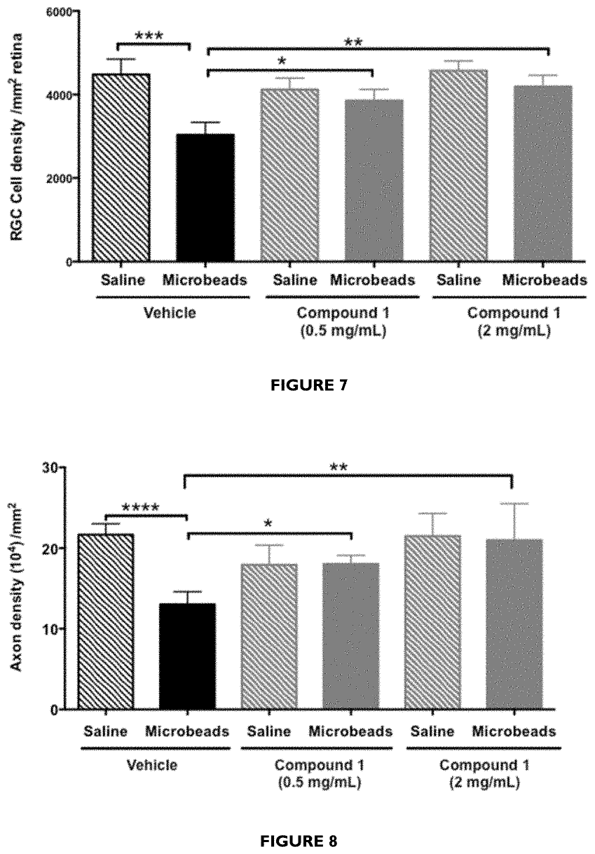

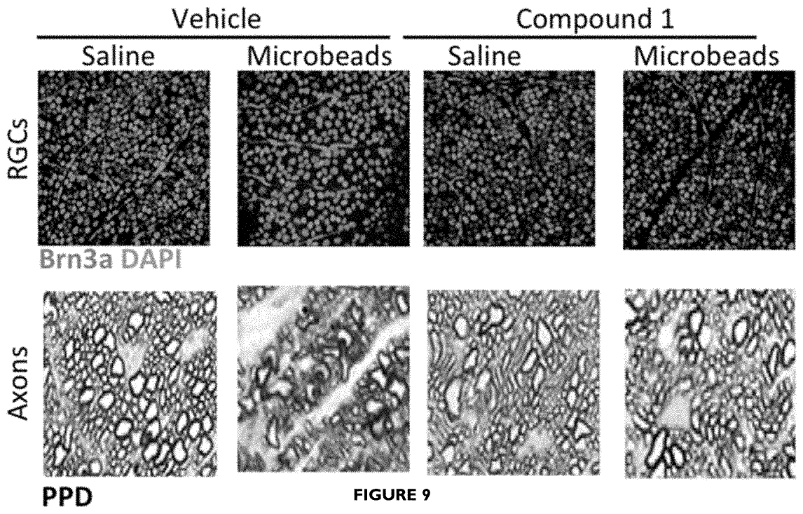

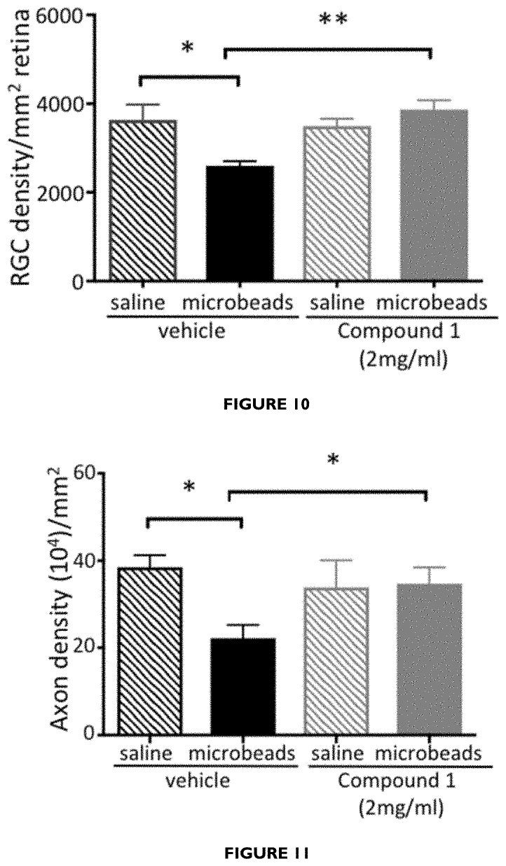

Only the 2.0 mg/mL (2 μg) dose of Compound 1 was tested at Day 7 post-microbead injection and, also, achieved nearly total preservation of retinal ganglion cell and axon density when compared to saline plus vehicle controls, as shown in the FIGS. 9, 10 and 11.

Inflammatory Microenvironment

Compound 1 inhibited microglial/macrophage activation.

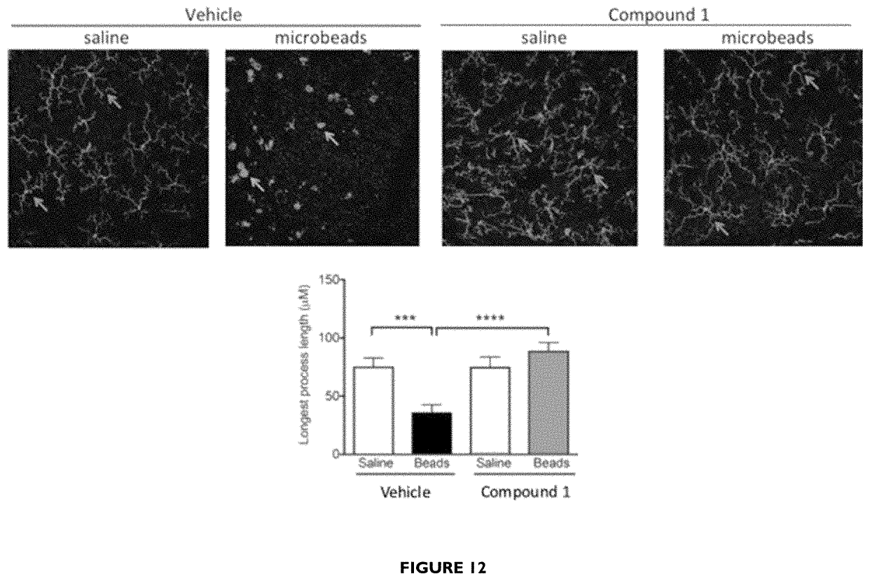

FIG. 12 depicts representative confocal images of retinal whole mounts at 28 days post microbead injection from mice treated with ONL1204 (or vehicle) at Day 0. Retinal whole mounts were stained with Ibal (microglia/macrophage). Yellow arrows indicate homeostatic microglia with dendritic morphology; blue arrows indicate activated microglia and/or infiltrating macrophages with amoeboid morphology. Morphometric analysis was performed on Ibal+cells in the ganglion cell layer (60 cells per retina) and the longest process length measured from the edge of the cell body (in μm) was used to quantitate microglia activation.

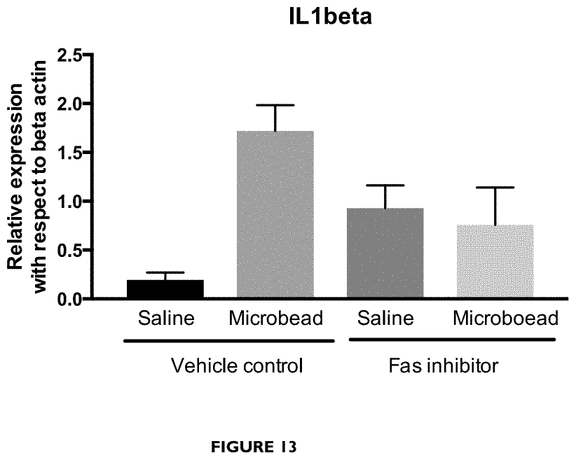

As shown in FIG. 13, additionally, treatment with Compound 1 substantially inhibited TNF-α, CCL2/MCP-1 and CCL3/MIP-1α gene expression and reduced the production of mature IL-1β. Quantitative PCR was performed on neural retina isolated at 28 days post microbead injection from mice treated with ONL1204 (or vehicle) on Day 0. Data shown are mRNA expression fold change over saline controls+/−SEM. N=4/group, *P<0.05, **P<0.01. For analysis of mature IL-1β (p17) production, protein lysates (20 μg per sample) were prepared from posterior eye cups (neural retina, choroid, and sclera) and analyzed by Western blot. Densitometry reveals a nearly two-fold reduction in the production of mature IL-1β following microbead injection in the mice treated with Compound 1 as compared to vehicle.

Conclusions:

Based on previous showing that Fas/FasL pathway is required for death of RGCs and loss of axons in the microbead-induced mouse model of glaucoma and the role of Fas in this process, as well as previous data from our laboratory showing protection of retinal cells following treatment with Compound 1, we were interested in determining whether Compound 1 could be used as a neuroprotective therapy to protect RGCs and prevent loss of axons in the microbead model of glaucoma.

These data demonstrate that treatment with Compound 1, a small peptide inhibitor of Fas, protects RGCs and prevents axon loss in this model of elevated IOP. This protection is observed even when Compound 1 is delivered after IOP has been elevated, which is a more clinically relevant scenario.

Furthermore, since Fas is known to trigger inflammatory signaling that can lead to additional cell death and tissues damage, the effect of Compound 1 on the inflammatory microenvironment was assessed. Treatment with Compound 1 reduced the inflammatory microenvironment, as indicated by the decreased expression of inflammatory cytokines/chemokines and the reduced number of activated microglia/macrophages. These data complement the company's separate efforts showing that treatment with Compound 1 results in decreased inflammatory markers.

These data support the potential of Compound 1 and Fas inhibition as part of a therapeutic strategy in the treatment of glaucoma.

Throughout this specification, various indications have been given as to preferred and alternative embodiments of the invention. However, the foregoing detailed description is to be regarded as illustrative rather than limiting and the invention is not limited to any one of the provided embodiments. It should be understood that it is the appended claims, including all equivalents, are intended to define the spirit and scope of this invention.

1-20. (canceled)

21. A method for preventing, treating or ameliorating an inflammation-mediated and/or complement-mediated disease or condition in a subject comprising administering to the subject a Fas inhibitor selected from the group consisting of Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor, in an amount effective to inhibit Fas signaling, and thereby prevent, treat or ameliorate the inflammation-mediated and/or complement-mediated disease or condition in the subject.
